(19) United States
(12) Patent Application Publication
(10) Pub. No.: US 2022/0401182 A1
(43) Pub. Date: Dec. 22, 2022

(54) SYSTEMS AND METHODS FOR DENTAL TREATMENT

(71) Applicant: ALIGN TECHNOLOGY, INC., San Jose, CA (US)

(72) Inventors: Kristin WINTERHALTER, Apex, NC (US); Eric BROWN, San Jose, CA (US); Sanket DESAI, San Jose, CA (US)

(73) Assignee: ALIGN TECHNOLOGY, INC., San Jose, CA (US)

(21) Appl. No.: 17/807,097

(22) Filed: Jun. 15, 2022

Related U.S. Application Data
(60) Provisional application No. 63/202,657, filed on Jun. 18, 2021.
(60) Provisional application No. 63/202,576, filed on Jun. 16, 2021.

(51) Int. Cl. A61C 7/00; A61C 13/34; G16H 20/40

(52) U.S. Cl. CPC A61C 7/002; A61C 13/34; G16H 20/40; A61C 7/08

(57) ABSTRACT

A method for preparing for and providing dental treatment may include determining, by an action management system, that 3D scan data of a patient's dentition has been received by a treatment planning system. A treatment planning system may generate a treatment plan to move the patient's teeth from an initial position towards a final position, based on the 3D scan of the patient's dentition. The treatment plan may be provided to a dental professional system. The action management system my generate an object for display on the dental professional system indicating that the treatment plan has been provided. An indication that the treatment plan is approved for fabrication may be received, and instructions to fabricate a dental appliance based on the treatment plan may be sent.

### RELATED APPLICATIONS

This application claims the benefit under 35 U.S.C. § 119(e) of U.S. Provisional Patent Application No. 63/202,657, filed Jun. 18, 2021, and of U.S. Provisional Patent Application No. 63/202,576, filed Jun. 16, 2021, the disclosures of which are incorporated, in their entirety, by this reference.

### BACKGROUND

Treating a patient with orthodontic and other dental treatments may involve series of actions carried out by a dental professional such as bringing in a new patient, providing the patient with treatment options, scanning the patient's teeth, developing a treatment plan, modifying and reviewing the treatment plan, fabricating appliances, fabricating restorative objects such as veneers, crowns, bridges, implants, and applying the appliances or restorative objects to the patient's dentition.

Orthodontic and dental treatments using a series of patient-removable appliances (e.g., “aligners”) are very useful for treating patients, and in particular for treating malocclusions. Treatment planning is typically performed in conjunction with the dental professional (e.g., dentist, orthodontist, dental technician, etc.), by generating a model of the patient's teeth in a final configuration and then breaking the treatment plan into a number of intermediate stages (steps) corresponding to incremental movements of the patient's teeth from an initial position towards the final position. Individual appliances are worn sequentially to move the teeth in each stage. This process may be interactive, adjusting the staging and in some cases the final target position, based on constraints on the movement of the teeth and the dental professional's preferences. Once the final treatment plan is finalized, the series of aligners may be manufactured based on the treatment plan.

Current systems and methods for treating patients are less than ideal in a number of ways. For example, current systems and methods may be slow to carry out the actions for treating the patient or may be unknowingly waiting on previously completed actions before proceeding with the next in the series of actions for treating the patient. Such events may lead to longer treatment times and delays in providing treatment to the patient.

The methods and apparatuses described herein may improve treatment, including potentially increasing the speed at which treatment plans may be completed and appliances provided to patients, as well as providing greater choices and control to the dental professional and/or the patient.

### SUMMARY

As will be described in greater detail below, the present disclosure describes various systems and methods for improving treatment, including increasing the speed at which treatment plans may be completed and appliances provided to patients, as well as providing greater choices and control to the dental professional and/or the patient.

In addition, the systems and methods described herein may improve the functioning of a computing device by reducing computing resources and overhead for acquiring and storing updated patient data, thereby improving processing efficiency of the computing device over conventional approaches. These systems and methods may also improve the field of orthodontic treatment by analyzing data to efficiently improve the treatment process and providing patients with access to more practitioners than conventionally available. The systems and methods may also improve patient compliance and shorten the overall treatment time.

### INCORPORATION BY REFERENCE

All patents, applications, and publications referred to and identified herein are hereby incorporated by reference in their entirety and shall be considered fully incorporated by reference even though referred to elsewhere in the application.

### BRIEF DESCRIPTION OF THE DRAWINGS

A better understanding of the features, advantages and principles of the present disclosure will be obtained by reference to the following detailed description that sets forth illustrative embodiments, and the accompanying drawings of which:

FIG. 1A illustrates an exemplary tooth repositioning appliance or aligner that can be worn by a patient in order to achieve an incremental repositioning of individual teeth in the jaw, in accordance with some embodiments.

FIG. 1B illustrates a tooth repositioning system, in accordance with some embodiments.

FIGS. 2A and 2B illustrate embodiments of a system for planning an orthodontic treatment, in accordance with some embodiments.

FIG. 3 shows steps in a dental treatment process, in accordance with some embodiments.

FIG. 4 shows steps in a dental treatment process, in accordance with some embodiments.

FIG. 5 shows steps in a dental treatment process, in accordance with some embodiments.

FIGS. 6A, 6B, and 6C show steps in a dental treatment process, in accordance with some embodiments.

FIG. 7 shows the display of customer and patient cards in organized columns, in accordance with some embodiments.

FIG. 8 shows steps in a dental treatment process, in accordance with some embodiments.

FIG. 9 shows a block diagram of an example computing system capable of implementing one or more embodiments described and/or illustrated herein, in accordance with some embodiments.

FIG. 10 shows a block diagram of an example computing network capable of implementing one or more of the embodiments described and/or illustrated herein, in accordance with some embodiments.

### DETAILED DESCRIPTION

The following detailed description and provides a better understanding of the features and advantages of the inventions described in the present disclosure in accordance with the embodiments disclosed herein. Although the detailed description includes many specific embodiments, these are provided by way of example only and should not be construed as limiting the scope of the inventions disclosed herein.

The present disclosure provides systems and methods for managing dental treatment. The systems and methods provided herein may improve the functioning of a computing device by efficiently updating and propagating data to various independent systems or providing links to data therebetween without requiring extraneous inter-system communications. In addition, the systems and methods provided herein may improve the field of medical care by improving a digital work flow procedure by reducing costs of human time spent on processing, increasing efficiency via automation, and reducing potential errors. Moreover, the systems and methods provided herein may improve the field of customer relationship management by improving compatibility between consumer-facing and professional applications and providing improved linking of customer data.

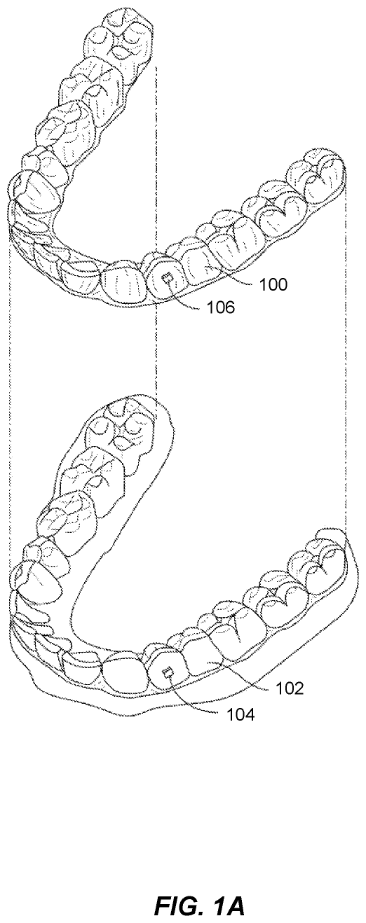

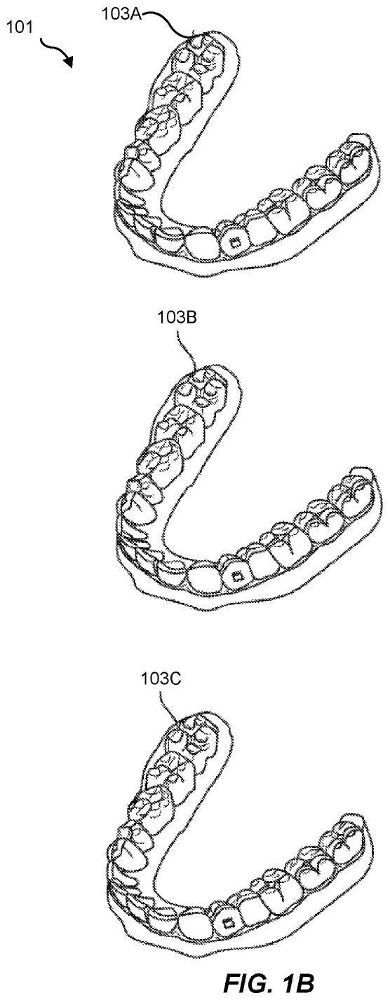

FIG. 1A illustrates an exemplary tooth repositioning appliance 100, such as an aligner that can be worn by a patient in order to achieve an incremental repositioning of individual teeth 102 in the jaw. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. The physical model (e.g., physical mold) of teeth can be formed through a variety of techniques, including 3D printing. The appliance can be formed by thermoforming the appliance over the physical model. In some embodiments, a physical appliance is directly fabricated, e.g., using additive manufacturing techniques, from a digital model of an appliance. In some embodiments, the physical appliance may be created through a variety of direct fabrication techniques, such as 3D printing. An appliance can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some or most, and even all, of the teeth will be repositioned at some point during treatment. Teeth that are moved can also serve as a base or anchor for holding the appliance as it is worn by the patient. In some embodiments, no wires or other means will be provided for holding an appliance in place over the teeth. In some cases, however, it may be desirable or necessary to provide individual attachments or other anchoring elements 104 on teeth 102 with corresponding receptacles or apertures 106 in the appliance 100 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted attachments suitable for use with orthodontic appliances are also described in patents and patent applications assigned to Align Technology, Inc., including, for example, U.S. Pat. Nos. 6,309,215 and 6,830,450.

FIG. 1B illustrates a tooth repositioning system 101 including a plurality of appliances 103A, 103B, 103C. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 101 can include a first appliance 103A corresponding to an initial tooth arrangement, one or more intermediate appliances 103B corresponding to one or more intermediate arrangements, and a final appliance 103C corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

Optionally, in cases involving more complex movements or treatment plans, it may be beneficial to utilize auxiliary components (e.g., features, accessories, structures, devices, components, and the like) in conjunction with an orthodontic appliance. Examples of such accessories include but are not limited to elastics, wires, springs, bars, arch expanders, palatal expanders, twin blocks, occlusal blocks, bite ramps, mandibular advancement splints, bite plates, pontics, hooks, brackets, headgear tubes, springs, bumper tubes, palatal bars, frameworks, pin-and-tube apparatuses, buccal shields, buccinator bows, wire shields, lingual flanges and pads, lip pads or bumpers, protrusions, divots, and the like. In some embodiments, the appliances, systems and methods described herein include improved orthodontic appliances with integrally formed features that are shaped to couple to such auxiliary components, or that replace such auxiliary components.

In some cases, after a patient has gone through treatment (e.g., primary order), the patient may require a second treatment (e.g., secondary order). When the doctor creates the secondary order, the doctor may request to remove some of the attachments placed in the primary order from the corresponding three-dimensional (3D) model of the patient's dental structure. The doctor may need to determine which attachments should be physically removed, which attachments should be added, and which ones should be left on the patient's teeth before starting the second treatment so that the appliances for the secondary order properly fit. However, the 3D model (e.g., as used with the primary order) may need to be updated for the secondary order.

A “dental consumer,” as used herein, may include a person seeking assessment, diagnosis, and/or treatment for a dental condition (general dental condition, orthodontic condition, endodontic condition, condition requiring restorative dentistry, etc.). A dental consumer may, but need not, have agreed to and/or started treatment for a dental condition. A “dental patient” (used interchangeably with patient herein) as used herein, may include a person who has agreed to diagnosis and/or treatment for a dental condition. A dental consumer and/or a dental patient, may, for instance, be interested in and/or have started orthodontic treatment, such as treatment using one or more (e.g., a sequence of) aligners (e.g., polymeric appliances having a plurality of tooth-receiving cavities shaped to successively reposition a person's teeth from an initial arrangement toward a target arrangement). Additionally or alternatively, a dental patient may, for instance, be interested in restorative treatment on one or more of their teeth.

A “dental professional” (used interchangeably with dentist, orthodontist, and doctor herein) as used herein, may include any person with specialized training in the field of dentistry, and may include, without limitation, general practice dentists, orthodontists, dental technicians, dental hygienists, etc. A dental professional may include a person who can assess, diagnose, and/or treat a dental condition. “Assessment” of a dental condition, as used herein, may include an estimation of the existence of a dental condition. An assessment of a dental condition need not be a clinical diagnosis of the dental condition. In some embodiments, an “assessment” of a dental condition may include an “image based assessment,” that is an assessment of a dental condition based in part or on whole on photos and/or images (e.g., images that are not used to stitch a mesh or form the basis of a clinical scan) taken of the dental condition. A “diagnosis” of a dental condition, as used herein, may include a clinical identification of the nature of an illness or other problem by examination of the symptoms. “Treatment” of a dental condition, as used herein, may include prescription and/or administration of care to address the dental conditions. Examples of treatments to dental conditions include prescription and/or administration of brackets/wires, clear aligners, and/or other appliances to address orthodontic conditions, prescription and/or administration of restorative elements to address bring dentition to functional and/or aesthetic requirements, etc.

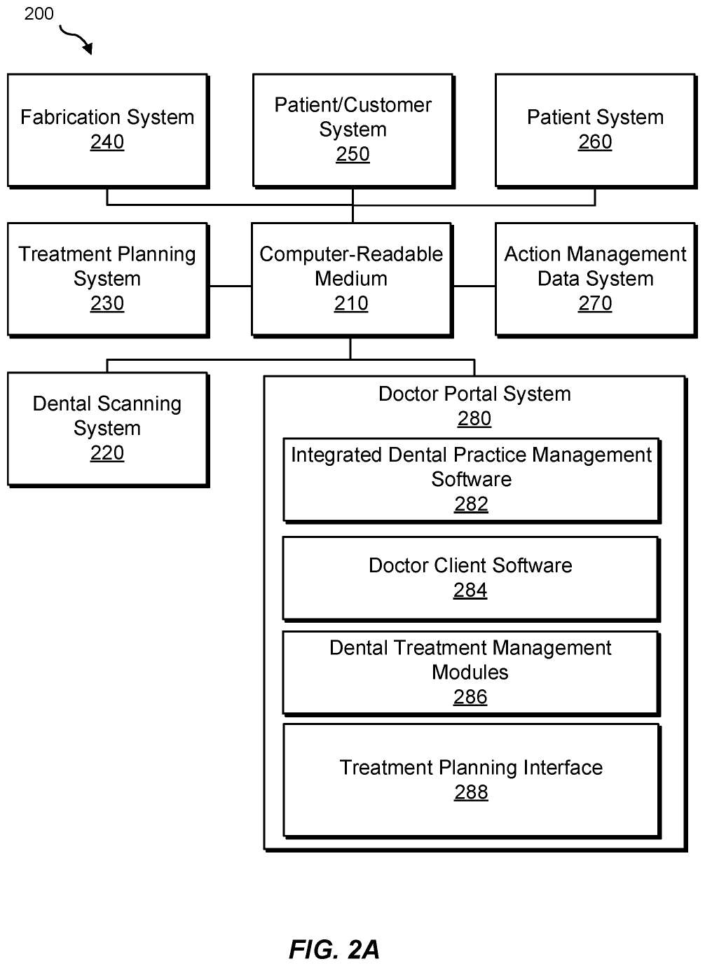

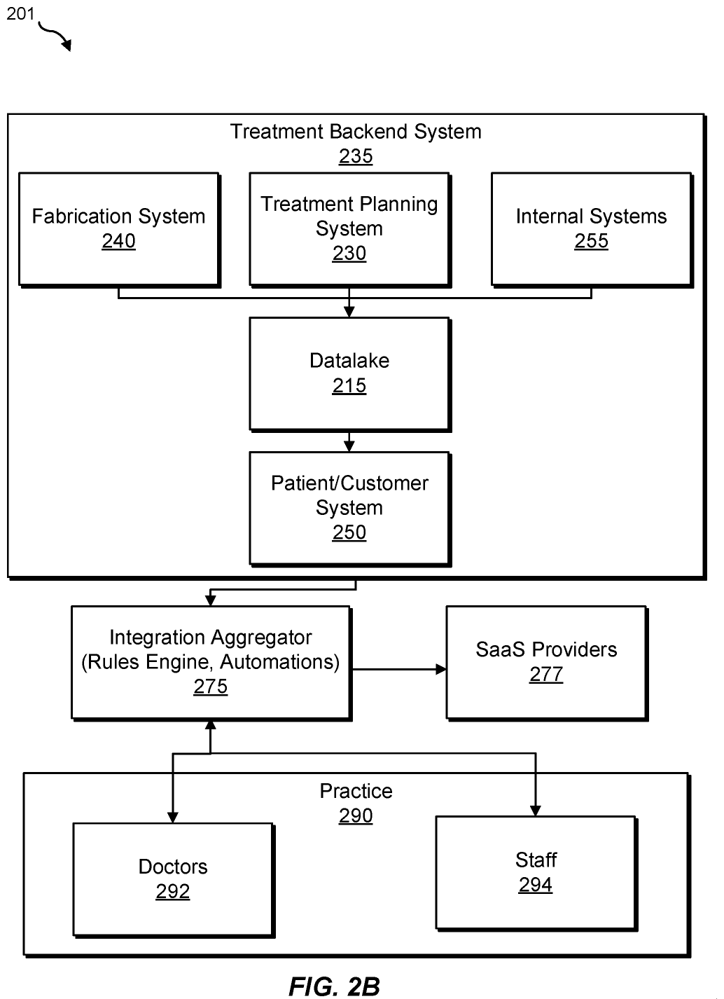

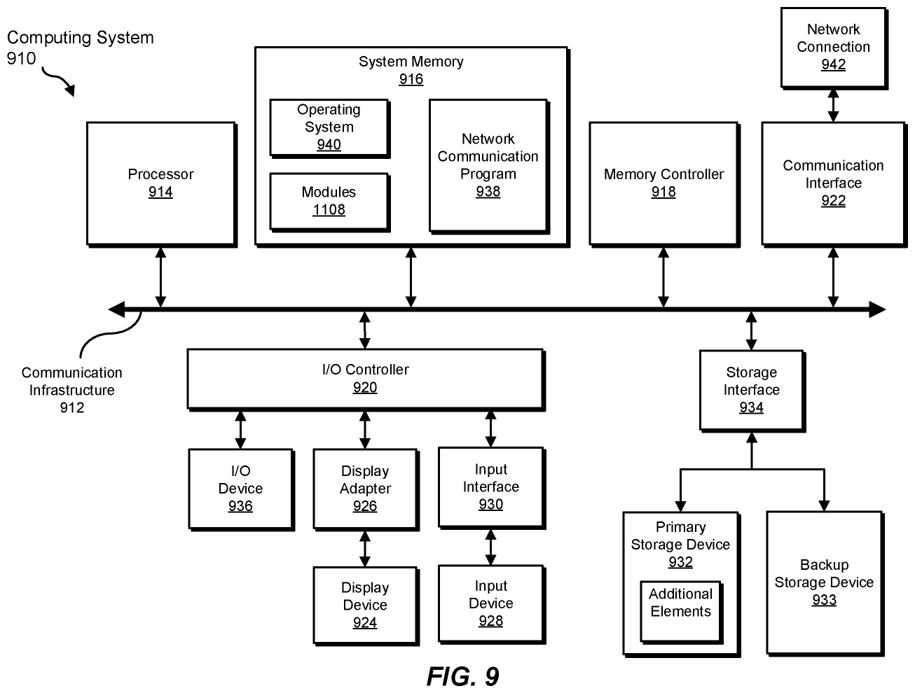

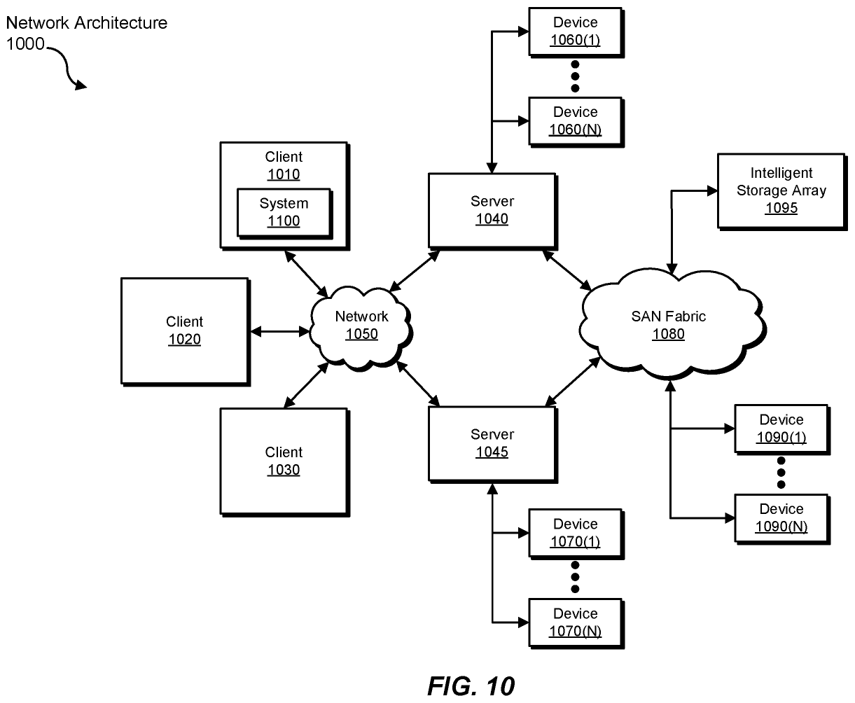

FIG. 2A shows a system 200 for simulating and planning an orthodontic treatment, in accordance with some embodiments. In the example of FIG. 2A, the system 200 includes a computer-readable medium 210, a dental scanning system 220, a dental treatment planning system 230, a fabrication system 240, a patient and customer data system 250, a patient system 260, a task management data system 270, and a dental professional system 280. One or more of the elements of the system 200 may include elements of such as those described with reference to the system shown in FIGS. 9 and 10 and vice versa. One or more elements of system 200 may also include one or more computer readable media including instructions that when executed by a processor, for example, a processor of any of systems 210, 220, 230, 240, 250, 260, 270, and 280 cause the respective system or systems to perform the processes described herein.

Dental scanning system 220 may include a computer system configured to capture one or more scans of a patient's dentition. Dental scanning system 220 may include a scan engine for capturing 2D or 3D images of a patient. Such images may include images of the patient's teeth, face, and jaw, for example. The images may also include x-rays, computed tomography, magnetic resonance imaging (MRI), cone beam computed tomography (CBCT), cephalogram images, panoramic x-ray images, digital imaging and communication in medicine (DICOM) images, or other subsurface images of the patient. The scan engine may also capture 3D data representing the patient's teeth, face, gingiva, or other aspects of the patient.

Dental scanning system 220 may also include a 2D imaging system, such as a still or video camera, an x-ray machine, or other 2D imager. In some embodiments, dental scanning system 220 may also include a 3D imager, such as an intraoral scanner, an impression scanner, a tomography system, a cone beam computed tomography (CBCT) system, or other system as described herein, for example. Dental scanning system 220 and associated engines and imagers can be used to capture the model data for use in detecting attachments, as described herein. Dental scanning system 220 and associated engines and imagers can be used to capture the 2D and 3D images of a patient's face and dentition for use in building a 3D parametric model of the patient's teeth as described herein. Examples of parametric models of the patient's teeth suitable for incorporation in accordance with the present disclosure are describe in U.S. application Ser. No. 16/400,980, filed on May 1, 2019, entitled “Providing a simulated outcome of dental treatment on a patient,” published as US20200000551 on Jan. 2, 2020, the entire disclosure of which is incorporated herein by reference.

Dental treatment planning system 230 may include a computer system configured to implement treatment plans. Dental treatment planning system 230 may include a rendering engine and interface for visualizing or otherwise displaying the simulated outcome of the dental treatment plan. For example, the rendering engine may render the visualizations of the 3D models described herein. Dental treatment planning system 230 may also determine an orthodontic treatment plan for moving a patient's teeth from an initial position, for example, based in part on the 2D image of the patient's teeth, to a final position. Dental treatment planning system 230 may be operative to provide for image viewing and manipulation such that rendered images may be scrollable, pivotable, zoomable, and interactive. Dental treatment planning system 230 may include graphics rendering hardware, one or more displays, and one or more input devices. Some or all of dental treatment planning system 230 may be implemented on a personal computing device such as a desktop computing device or a handheld device, such as a mobile phone. In some embodiments, at least a portion of dental treatment planning system 230 may be implemented on a scanning system, such as dental scanning system 220.

Fabrication system 240 may include direct fabrication systems and indirect fabrication systems. The fabrication system 240 may directly fabricate a dental appliance such as an aligner, crown, or bridge through additive or subtractive manufacturing or other direct fabrication processes. The fabrication system 240 may indirectly fabricated a dental appliance such as an aligner, crown, or bridge by first fabricating a mold for the aligner, crown, or bridge and then forming the aligner, crown, or bridge or other orthodontic device on or in the mold.

Patient and customer data system 250 may be or include a customer management system. The patient and customer data system 250 may include data relevant to the patient for example their name, age, contact information, dental records, treatment records, and other information to identify and treat the patient. The patient and customer data system 250 may also include a non-transitory computer readable medium comprising instructions for accessing and causing other portions of the system 200 to execute actions or tasks. For example, in some embodiments the passenger and customer data system 250 may execute instructions to use an API to cause the action management system to carry out one or more steps in processes described herein. For example, to cause the action management system to find a doctor's practice and create a new object to pass customer information to the dental professional system. In some embodiments, the patient and customer data system 250 may interface with other system such as the treatment planning system to determine the status of treatment plan generation, in some embodiments, the patient and customer data system may interface with the fabrication system to determine the status of dental appliance fabrication.

Patient systems 260 may include one or more systems used by the patient for treatment. For example, a mobile device including hardware and/or software modules for capturing images of the patient's teeth and tracking treatment, advising the patient of treatment progress, facilitating communication between the patient and the dental professional, and receiving treatment. Patient systems 260 may include any computing device associated with a patient and may execute software such as lead generation software, smile visualization software, appliance finance software, and/or software to access their treatment plan.

Action management data system 270 may include modules for receiving inputs from the other systems or querying the other systems to determine a current status of a patient's treatment and providing the next treatment actions to the dental professional and/or patient. The action management data system 270 may also include a non-transitory computer readable medium comprising instructions for accessing and causing other portions of the system 200 to execute actions or tasks or to update other portions of the system 200 such as the data residing in other portions of the system 200. For example, in some embodiments the action management data system 270 may execute instructions to use an API to cause the action management system to carry out one or more steps in processes described herein. For example, to create a new object for visual display on the dental professional system, to update an aspect of the dental practice management system, to generate and send correspondence including forms and other information to patients, doctors, treatment providers or other entities. In some embodiments, the action management data system 270 may interface with other system such as the treatment planning system to determine the status of treatment plan generation, in some embodiments, the patient and customer data system may interface with the fabrication system to determine the status of dental appliance fabrication.

The dental professional system 280 may be an integrated dental system and may include modules for managing and treating patients. In some embodiments, the dental professional system 280 may include an integrated dental practice management software module 282, a doctor-client software module 284, the treatment planning interface 288, and other dental treatment management modules 286.

The integrated dental practice management software module 282 may include a computer system or module that can interface with the other systems, such as through the implemention of APIs, that connect the data within the patient customer data system 250 with the data and modules within the task management data system 270 and other systems described herein and can configure one or more actions that a doctor can interact with through the integrated dental system and/or other software on the dental professional system.

The doctor-client software module 284 can include lead generation software, patient financing software, and/or non-clinical software used as part of the doctor's practice.

The treatment planning interface 288 may include a computer system configured to review and modify treatment plans. Dental treatment planning interface 288 may include a rendering engine and interface for visualizing or otherwise displaying the simulated outcome of the dental treatment plan. For example, the rendering engine may render the visualizations of the 3D models described herein. Dental treatment planning interface 288 may be operative to provide for image viewing and manipulation such that rendered images may be scrollable, pivotable, zoomable, and interactive. Dental treatment planning interface 288 may include graphics rendering hardware, one or more displays, and one or more input devices. Some or all of dental treatment planning interface 288 may be implemented on a personal computing device such as a desktop computing device or a handheld device, such as a mobile phone. In some embodiments, at least a portion of dental treatment planning interface 288 may be implemented on a scanning system, such as dental scanning system 220. In some embodiments, at least a portion of dental treatment planning interface 288 may be implemented on the treatment planning system, such as treatment planning system 230.

FIG. 2B shows a system 201 for simulating and planning an orthodontic treatment, in accordance with some embodiments. The features and benefits of the system 201 may include the same features and benefits of system 200, depicted in FIG. 2A. In the example of FIG. 2B, the system 201 includes treatment backend system 235, a practice system 290, integration aggregator system 275, and SaaS provider systems 277.

The treatment back in system 235 may include a dental treatment planning system 230, a fabrication system 240, a data lake 215, an action management data system 270, and other internal systems 255. The practice system 290 which may be similar to the dental professional system 280 may include a doctor's system 292, and a staff system 294.

One or more of the elements of the system 201 may include elements of such as those described with reference to the system shown in FIGS. 9 and 10 and vice versa. One or more elements of system 201 may also include one or more computer readable media including instructions that when executed by a processor, for example, a processor of any of the systems 215, 230, 240, 255, 270, 275, 277, 290, 292, and 294 cause the respective system or systems to perform the processes described herein.

The data lake system 215 may be a centralized repository for storing structured and unstructured data. The data lake system 215 may store relational and non-relational databases, treatment plan data, patient data, and any other data used by the systems 200, 201. Any of the other systems described herein may store, retrieve, manipulate, query, or otherwise use the data stored in the data lake system 215 for use in treating a patient.

System 201 may include a dental treatment planning system 230 similar to the dental treatment planning system 230 discussed with respect to FIG. 2A. The dental treatment planning system 230 may include a computer system configured to implement treatment plans. Dental treatment planning system 230 may include a rendering engine and interface for visualizing or otherwise displaying the simulated outcome of the dental treatment plan. For example, the rendering engine may render the visualizations of the 3D models described herein. Dental treatment planning system 230 may also determine an orthodontic treatment plan for moving a patient's teeth from an initial position, for example, based in part on the 2D image of the patient's teeth, to a final position. Dental treatment planning system 230 may be operative to provide for image viewing and manipulation such that rendered images may be scrollable, pivotable, zoomable, and interactive. Dental treatment planning system 230 may include graphics rendering hardware, one or more displays, and one or more input devices. Some or all of dental treatment planning system 230 may be implemented on a personal computing device such as a desktop computing device or a handheld device, such as a mobile phone. In some embodiments, at least a portion of dental treatment planning system 230 may be implemented on a scanning system, such as the dental scanning system 220.

The backend system 235 may also include other internal systems 255 for use in providing dental treatment of patients.

The backend system 235 may also include a patient and customer data system, such as patient customer data system 250 may be or include a customer management system. The patient and customer data system 250 may include data relevant to the patient for example their name, age, contact information, dental records, treatment records, and other information to identify and treat the patient. The patient and customer data system 250 may also include a non-transitory computer readable medium comprising instructions for accessing and causing other portions of the system 200 to execute actions or tasks. For example, in some embodiments the patient and customer data system 250 may execute instructions to use an API to cause the action management system to carry out one or more steps in processes described herein. For example, to cause the action management system to find a doctor's practice and create a new object to pass customer information to the dental professional system. In some embodiments, the patient and customer data system 250 may interface with other system such as the treatment planning system to determine the status of treatment plan generation, in some embodiments, the patient and customer data system may interface with the fabrication system to determine the status of dental appliance fabrication.

The backend system 235 may also include a fabrication system, such as fabrication system 240 may include direct fabrication systems and indirect fabrication systems. The fabrication system 240 may directly fabricate a dental appliance such as an aligner, crown, or bridge through additive or subtractive manufacturing or other direct fabrication processes. The fabrication system 240 may indirectly fabricate a dental appliance such as an aligner, crown, or bridge by first fabricating a mold for the aligner, crown, or bridge and then forming the aligner, crown, or bridge or other orthodontic device on or in the mold.

The system 201 may also include an integrator aggregator system 275. The integrator aggregator system 275 may be similar to the action management system 270 and may be located separately from the backend system 235. In some embodiments, the integrator aggregator system 275 may include modules for receiving inputs from the other systems or querying the other systems to determine a current status of a patient's treatment and providing the next treatment actions to the dental professional and/or patient. The action management data system 270 may also include a non-transitory computer readable medium comprising instructions for accessing and causing other portions of the system 201 to execute actions or tasks or to update other portions of the system 201 such as the data residing in other portions of the system 201.

For example, in some embodiments the integrator aggregator system 275 may execute instructions to use an API to cause the integrator aggregator system 275 to carry out one or more steps in processes described herein. For example, to create a new object for visual display on the dental professional system, to update an aspect of the dental practice management system, to generate and send correspondence including forms and other information to patients, doctors, treatment providers or other entities. In some embodiments, the integrator aggregator system 275 may interface with other systems such as the treatment planning system to determine the status of treatment plan generation, in some embodiments, the patient and customer data system may interface with the fabrication system to determine the status of dental appliance fabrication.

In some embodiments, the system may include software-as-a-service (SaaS) provider modules 277. The SaaS provider modules may interface with one or more of the integration aggregator the practice 290 and the backend system 235 to provide user interfaces, automation, and other features.

The practice system 290 may be similar to the dental professional system 280. In some embodiments, the practice system 290 may include a doctor system 292 and a staff system 294. In some embodiments, the practice system 290 may be an integrated dental system and may include modules for managing and treating patients. In some embodiments, the dental professional system 280 may include an integrated dental practice management software module 282, a doctor-client software module 284, the treatment planning interface 288, and other dental treatment management modules 286. In some embodiments, the doctor system 292 includes the treatment planning interface 288 and the doctor-client software module 284. In some embodiments, the doctor system and the staff system contain portions of the integrated dental practice management software module 282. In some embodiments, the staff system includes other dental treatment manager module 286. In some embodiments, the doctor system 292 contains modules for managing the clinical treatment of patients while the staff system 294 include modules for managing patient relationships, patient data, practice management, billing, and non-treatment related modules.

In some embodiments, the doctor system 292 may include a dental scanning system 220 may include a computer system configured to capture one or more scans of a patient's dentition. Dental scanning system 220 may include a scan engine for capturing 2D or 3D images of a patient. Such images may include images of the patient's teeth, face, and jaw, for example. The images may also include x-rays, computed tomography, magnetic resonance imaging (MRI), cone beam computed tomography (CBCT), cephalogram images, panoramic x-ray images, digital imaging and communication in medicine (DICOM) images, or other subsurface images of the patient. The scan engine may also capture 3D data representing the patient's teeth, face, gingiva, or other aspects of the patient.

The integrated dental practice management software module 282 may include a computer system or module that can interface with the other systems, such as through the implemention of APIs, that connect the data within the patient customer data system 250 with the data and modules within the task management data system 270 and other systems described herein and can configure one or more actions that a doctor can interact with through the integrated dental system and/or other software on the dental professional system.

The doctor-client software module 284 can include lead generation software, patient financing software, and/or non-clinical software used as part of the doctor's practice.

The treatment planning interface 288 may include a computer system configured to review and modify treatment plans. Dental treatment planning interface 288 may include a rendering engine and interface for visualizing or otherwise displaying the simulated outcome of the dental treatment plan. For example, the rendering engine may render the visualizations of the 3D models described herein. Dental treatment planning interface 288 may be operative to provide for image viewing and manipulation such that rendered images may be scrollable, pivotable, zoomable, and interactive. Dental treatment planning interface 288 may include graphics rendering hardware, one or more displays, and one or more input devices. Some or all of dental treatment planning interface 288 may be implemented on a personal computing device such as a desktop computing device or a handheld device, such as a mobile phone. In some embodiments, at least a portion of dental treatment planning interface 288 may be implemented on a scanning system, such as dental scanning system 220. In some embodiments, at least a portion of dental treatment planning interface 288 may be implemented on the treatment planning system, such as treatment planning system 230.

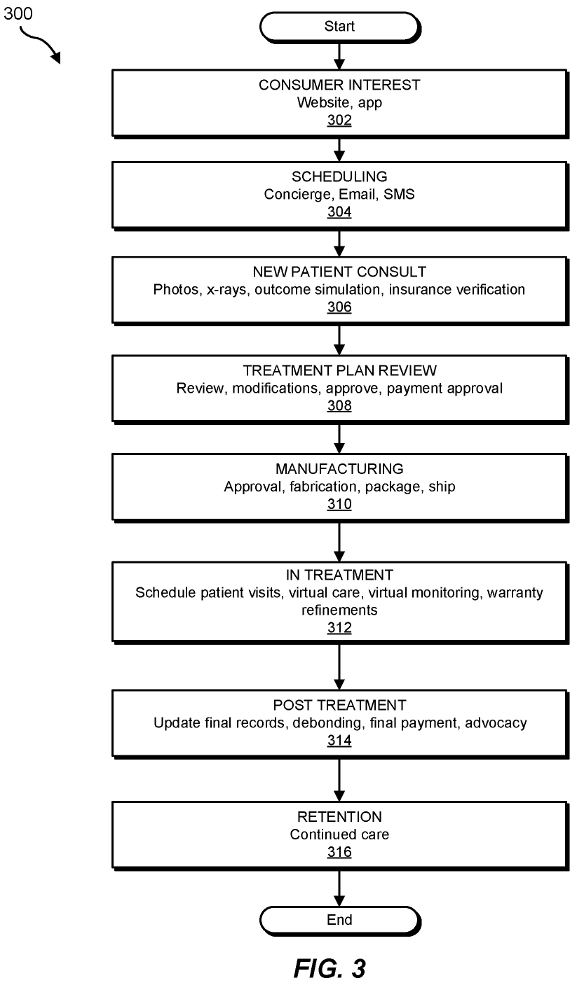

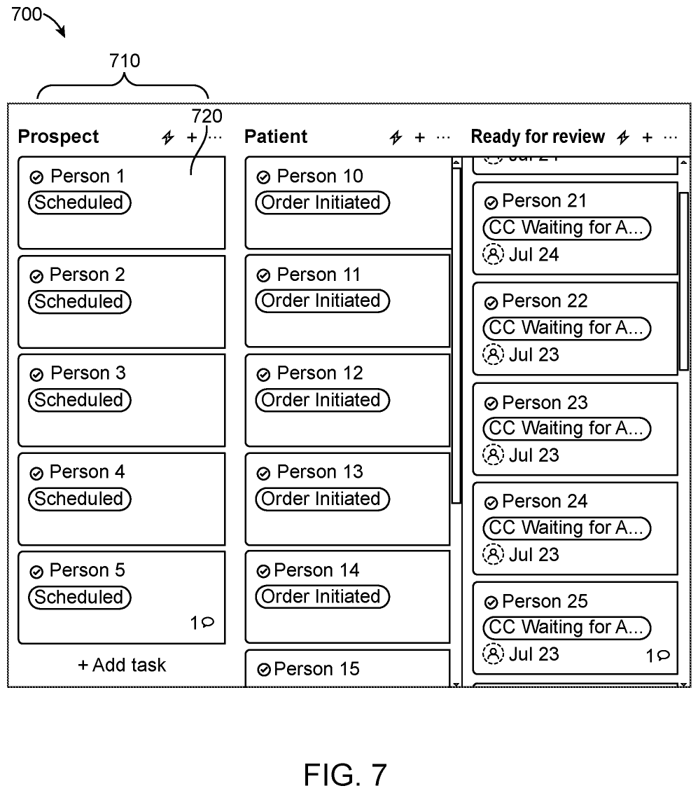

FIG. 3 shows the steps in a process 300 for treating a consumer. At the consumer interest block 302 a consumer may become aware of their dental or orthodontic treatment options and may contact a dental professional's practice through their website, through a consumer dental application, such as the My Invisalign App, or through a doctor locator website. At the consumer interest block, consumer data is received and stored in the patient and customer data system 250. Consumer data may include the information discussed above with respect to the patient customer data system 250. At the consumer interest block, the consumer's information may be provided to a doctor such as via the dental professional system 284, for display, such as shown in the left column of FIG. 7. In some embodiments, a dental professional, a patient, or other person may carry out one or more of the actions or tasks generated or provided in method 300 or any of the other methods described herein.

In some embodiments, a patient may directly contact a doctor to initiate treatment. In some embodiments, the dental professional system receives the customer data from the patient directly or from the patient through the dental professional or their staff. After receiving the customer data, the dental professional system may provide the customer data to the patient and customer data system 250. In some embodiments, the dental professional system also schedules the appointment and provides important information to the patient and customer data system 250. Upon receiving customer data in scheduling information, the process may proceed to block 306.

At the scheduling block 304, a consumer's (now a patient) and doctor's schedule is coordinated in order to find a mutually agreeable time and date for the patient and doctor to meet and begin treatment. In some embodiments, the customer patient module will call the action management system to find the doctor's practice and create a new object in the dental professional system that includes the patient data such as their name contact information and dental treatment information.

In some embodiments, at the scheduling block 304, the action management system may cause the object created at block 302 appear visually on the staff system 294. In some embodiments, the action management system may update the dental practice management system to include the patient data. In some embodiments, the action management system may coordinate the generation and sending of correspondence to the patient. In some embodiments, the correspondence may include electronic forms for collecting patient and treatment information. In some embodiments, the SaaS system will aid in providing the forms to the patient, such as through the patient system and collect the form information. Upon the patient's completion of the forms, the action management system may automatically receive the forms from the SaaS and update the information in the dental professional system based on the data in the forms. In some embodiments, the action management system may confirm insurance coverage.

At the new patient consult block 306, in response to the scheduling of an appointment, a series of actions may be generated for the patient and doctor to carry out during the new patient consultation. These actions may include actions of taking photos, x-rays, and three-dimensional scans of the patient's dentition in order to stimulate the patient's treatment, for example using treatment simulator. At the new patient counsel block, these actions may be sent to a doctor and such information may be provided to the dental professional system 284 for display for example, as shown in the center column of FIG. 7. In response to the generation of photos, x-rays and three-dimensional scans, actions may be indicated as complete. For example, the scanning system may provide the photos, x-rays, and three-dimensional scans to the treatment planning system and upon acknowledgment, the task management system 270 may indicate the actions are complete and generate new actions based on the status of the patient's treatment.

After completion of the actions at the new patient consult block 306, the treatment planning system may generate a treatment plan for treating the patient's dentition. Upon completion of the generation of an initial treatment plan, the treatment plan may be sent to the dental professional. The task management system may receive an indication that the treatment plan is complete and has been sent to the dental professional or is otherwise ready for the dental professional's review. Upon generation of the treatment plan or delivery of the treatment plan, the task management system may generate actions for the next steps in the patient's treatment. For example, the task management system may generate an action for reviewing the treatment plan. Such actions may be provided to the dental professional, for example via the dental professional system 284 for display, such as shown in the right column of FIG. 7.

In some embodiments, after completion of the actions at the new consult block 306, the treatment planning system may generate a plurality of treatment plan options for treating the patient's dentition. The treatment plan options may include treatment plans with different goals, such as different time frames for completion, the use or nonuse of various dental objects and appliances, and other orthodontic and restorative options. For example, a first treatment plan may correct only the upper arch or only the anterior teeth. By treating less than all of the patient's dentition, the treatment may be completed in a shorter amount of time as compared to a second treatment plan presented to the patient that corrects both the anterior and posterior teeth of both the upper and lower arches.

In some embodiments, a first of the plurality of treatment plans may use attachments to provide greater tooth positioning forces which may result a fewer number of treatment stages and a shorter overall treatment time for the patient. In some embodiments, the patient may prefer not to use attachments for cosmetic reasons. A second of the plurality of options may include a greater number of treatment stages, a longer treatment time, but not to use attachments in order to provide the patient with this option.

Each of the plurality of treatment plans may include different target positions of the teeth at the end of treatment. Each of the different target positions may be displayed to the patient along with information regarding the number of treatment stages, total treatment time, and other aspects of the dental treatment for selection by the patient. After selecting a treatment plan, the treatment plan may be sent to the doctor for final review, revision in approval. A dental professional or patient may carry out the actions.

Generating a treatment plan may include one or more steps, including receiving a digital representation of a patient's teeth and generating one or more treatment stages of a treatment plane to incrementally responding the patient's teeth from an initial position towards a final position.

The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

At the treatment plan review block 308, the dental professional may review the treatment plan and provide modifications and/or approve the treatment plan. The treatment plan review may be an iterative process wherein the dental professional reviews and modifies an initial treatment plan. Then, an updated treatment plan may be generated by the treatment planning system and sent back to the dental professional for further review and modifications. Multiple updated treatment plans may be generated and reviewed through this process. Once the dental professional is satisfied with the treatment plan the dental professional may approve of the treatment plan. The finalized treatment plan may be sent to the fabrication system for fabrication. In some embodiments, upon finalization of the treatment plan, the task management system may generate an action for fabricating the dental appliance.

At the manufacturing block 310, the fabrication system fabricates the dental appliance. The fabrication system 240 may directly fabricate a dental appliance such as an aligner or crown or bridge through additive or subtractive manufacturing or other direct fabrication processes. The fabrication system 240 may indirectly fabricate a dental appliance such as an aligner, crown, or bridge by first fabricating a mold for an aligner, crown, or bridge and then forming the aligner, crown, or bridge or other orthodontic device on or in the mold. At the manufacturing block, the task management system 270 may receive information from the fabrication system regarding the status of the fabrication of the dental appliances and, based on the status, generate actions for the dental professional and/or the patient and provide these actions for display on the dental professional system 280 and/or the patient system 260.

In step 1030, at least one orthodontic appliance is fabricated based on the generated treatment stages. For example, a set of appliances can be fabricated to be sequentially worn by the patient to incrementally reposition the teeth from the initial arrangement to the target arrangement. Some of the appliances can be shaped to accommodate a tooth arrangement specified by one of the treatment stages. Alternatively or in combination, some of the appliances can be shaped to accommodate a tooth arrangement that is different from the target arrangement for the corresponding treatment stage. For example, as previously described herein, an appliance may have a geometry corresponding to an overcorrected tooth arrangement. Such an appliance may be used to ensure that a suitable amount of force is expressed on the teeth as they approach or attain their desired target positions for the treatment stage. As another example, an appliance can be designed in order to apply a specified force system on the teeth and may not have a geometry corresponding to any current or planned arrangement of the patient's teeth.

At the in treatment block 312, the patient may regularly wear their dental appliances according to the schedule treatment determined by the treatment plan. At the in treatment block, the task management system 270 may use the treatment plan to generate actions for the doctor and/or the patient. For example, for orthodontic treatment with aligners, the task management system 270 may generate actions for the wearing of the aligners. For example, every two weeks the task management system may remind the patient to switch to a new aligner. In some embodiments, the task management system may also schedule patient visits and generate actions for virtual monitoring. For example, the task management system 270 may generate an action for the patient to take a two-dimensional image of their teeth. The task management system may, upon receiving an indication that the two-dimensional image of the teeth has been taken and provided to the dental professional, generate an action for the dental professional to review the two-dimensional image. The dental professional may carry out the actions.

After treatment is complete, at the post treatment block 314, the patient's final records may be updated and the task management system 270 may generate actions for finalizing treatment, such as debonding of attachments use during treatment. The test management system 270 may also generate actions for requesting and receiving final payment. The actions may be carried out.

After the post treatment block 316, and the retention block continued care may be provided to the patient. For example, in some embodiments a task management system 270 may generate actions for continued care for the patient. For example, continued virtual monitoring may continue for a period of time in order to check that the patient is not regressing. A dental professional or patient may carry out the actions.

In some embodiments, the dental professional may interface with the system by providing login information at, for example, a dental portal system, a scanner system, etc. of a dental professional system. The login information may be received by the dental portal system, scanner system, etc. of the dental professional system.

After receiving valid login information, customer and/or patient data from the patient and customer data system may be provided to the dental professional at the dental portal system, scanner system, etc. of a dental professional system. The data may be gathered through queries to the data or database within the patient and customer data system or provided to the dental professional system by the customer and patient data system.

In some embodiments the integrated dental practice management software may use the customer patient data to create dental practice management tasks for the consumers and/or patients. In some embodiments, this may be implemented using APIs. For example, the APIs may use the customer and patient data to determine the type of dental treatment to be provided to the patient. In some embodiments the dental practice management tasks may relate to orthodontic tasks, restorative task, or general dental tasks. In some embodiments, the dental practice management tasks may include lead generation tasks to convert a consumer to a patient. For example, the data may include reviewing the customer and patient data in order to determine whether the dental professional can treat the patient and address the patient's dental issues. In some embodiments, the actions may include verifying that the consumer has appropriate insurance to cover treatment costs. A dental professional or patient may carry out the actions.

In some embodiments, the actions may include providing patient data from the customer and patient data system to the treatment planning system. In some embodiments, the actions may include actions related to the staging and treatment of the patient's teeth to move the teeth from an initial position towards a final position.

In some embodiments, the actions may include reviewing a proposed treatment plan, revising or modifying the treatment plan, accepting or approving the treatment plan, and other tasks related to reviewing, submitting, and modifying treatment plan. A dental professional may carry out the actions.

In some embodiments the actions may include the directed scanning of a patient's dentition with the scanning system and/or sending scan data of the patient's dentition to the treatment planning system.

In some embodiments, the actions may include instructions for fabricating dental appliances as discussed herein. The dental appliances may be fabricated.

In some embodiments, actions may be displayed through task or project management and visualization systems through a second API. The second API may facilitate the display of scanning actions, lead generation actions, treatment plan generation actions, treatment plan review actions, fabrication actions, appointment management actions, and customer review actions. In some embodiments, the information may be received or processed through interactions with doctors regarding digital dental practice management tasks and the digital dental practice management tasks may be managed by the system based on the doctor's interactions. A dental professional or patient may carry out the actions.

In some embodiments, the generated actions may be represented visually for review by the dental professional and/or the patient. In some embodiments, the display may include touchscreen display elements for a touchscreen interface. In some embodiments, the actions may be displayed on a scanning system, such as the iTero scanning system, a doctor website, the patient system, and/or the dental professional system. The generated actions may be displayed in the form of cards, or in columns or swim lanes. In some embodiments, the tasks are automatically generated in response to receiving or verifying data generated by carrying out other, previously generated actions. For example, upon receiving a completed scan, treatment planning actions may be generated. A dental professional or patient may carry out the tasks.

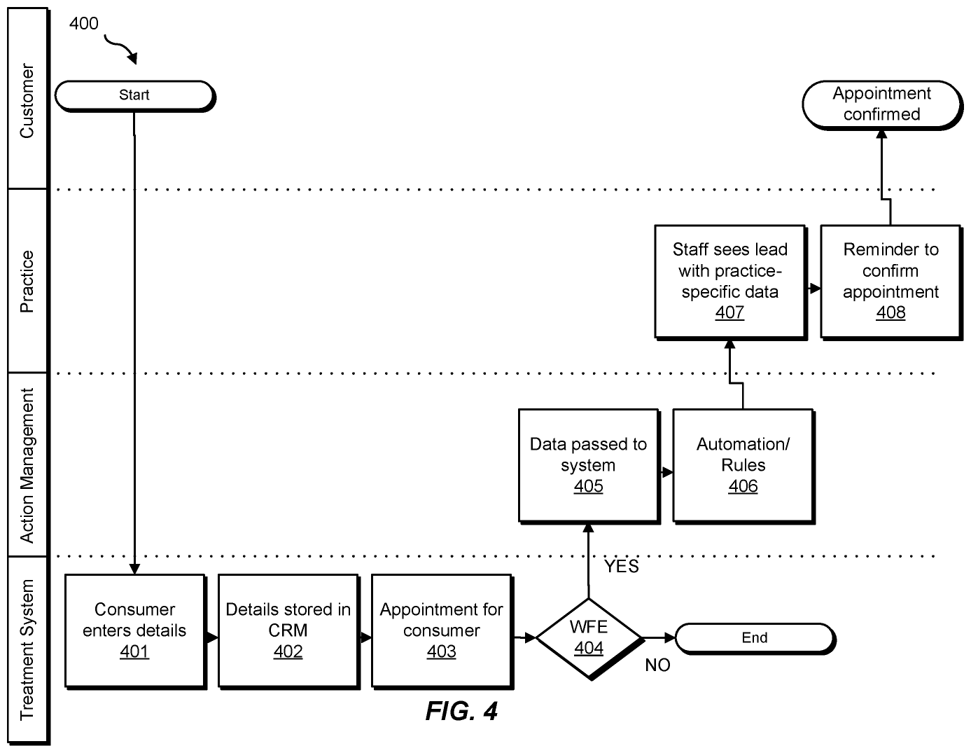

FIG. 4 shows a process 400 for initiating dental treatment of a patient. FIG. 4 includes four rows, the customer row, a practice row, an action management row, and a treatment system row. The location of a block in a particular row the case the systems that may carry out the actions of that block. For example, in the customer row the customer may use a patient system to interface with the other systems in order to carry out an action. In the practice row, the dental professional system may carry out the actions of the blocks. In the action management row, an action management system or integration aggregator system may carry out the actions of the blocks. In the treatment system row, the backend system may carry out the actions of the blocks.

At block 401, the patient or consumer enters their details into a customer and patient data system. The patient may use a form such as any form provided by a SaaS system to provide their information. At block 402, the SaaS system may provide the information to the customer patient data system. In some embodiments, an action management system they retrieve order received the patient information from the SaaS system and store that information in a customer relation management system, such as the customer patient data system. At block 403, in some embodiments, the backend system may schedule an appointment for the consumer, as discussed herein. In some embodiments upon schedule an appointment for the consumer a workflow enabled check may occur at block 404. The workflow enabled check may review the data in the customer and patient data system, the appointment information, and other information related to the patient and determine whether the information is appropriate involved. If so, the process continues otherwise the process ends.

At block 405, the customer data and the appointment information may be sent to the action management system. At block 405, the action management system receives the data and then at block 406 automated tasks are carried out based on rules and instructions executed by the action management system. As described herein, such automation and rules may configure the various systems described herein in order to automate actions or other tasks relating to the treatment plan. Automation and rules may include updating data, propagating the updated data to relevant applications and systems, establishing and propagating trigger events, etc. An end user or a system administrator may establish rules. In some examples, the rules may be “if/then” based. For example, a rule may state that if a predetermined amount of time has elapsed, an email may be sent to the patient. Another rule may state that if a specified event occurs, a message may be sent to the doctor. Automations may similarly apply trigger conditions and responses to various connected applications and/or systems.

For example, at block 406 the customer data in the appointment information is received as input for carrying out the rules and automation and the output of the rules and automation may be updates to the dental professional system including the generation of actions to be completed by the dental professional, and updates to data stored within the dental professional system such as treatment data patient data and other information.

At block 407, the practice management system displays the patient details provided by the consumer at block 401 and stored in the CRM at block 402. In some embodiments, the details may include the appointment information along with the patient information. In response to the display of the patient details, at block 408, the staff may initiate, through the dental professional system, a reminder communication to be sent to the patient to confirm the appointment.

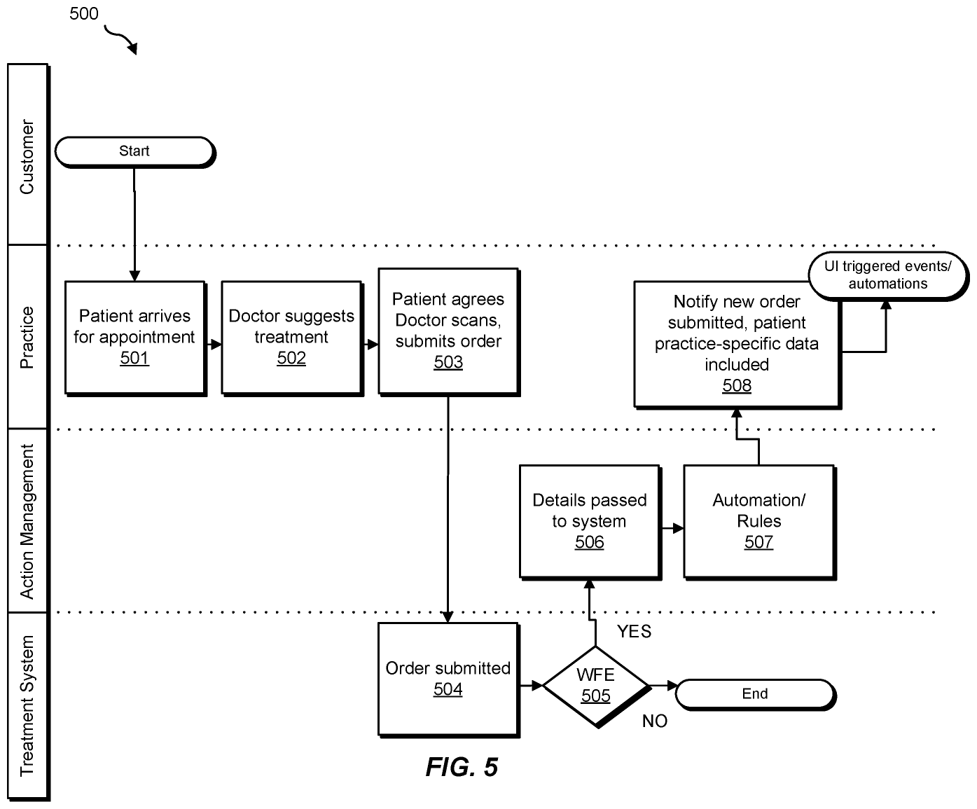

FIG. 5 is a flow diagram of an example process 500 for a new order, in accordance with some embodiments. As shown in FIG. 5, process 500 may include interactions between a customer (e.g., a prospective customer, a patient, etc.), a practice (e.g., a doctor and/or other dental practitioner and corresponding staff, and an office for the practice), an action management system (e.g., action management data system 270), and a treatment system (e.g., treatment backend system 235). The location of a block in a particular row the case the systems that may carry out the actions of that block. For example, in the practice row, the dental professional system may carry out the actions of the blocks. In the action management row, an action management system or integration aggregator system may carry out the actions of the blocks. In the treatment system row, the backend system may carry out the actions of the blocks.

At block 501, a patient may arrive for an appointment with a doctor, which may have been previously scheduled as further described herein. The patient may arrive at the practice and discuss the patient's issues with the doctor. At block 502, the doctor may suggest a treatment.

At block 503, the patient may agree to the treatment. The doctor may scan (using, e.g., dental scanning system 220) the patient's dentition as further described herein. The doctor may then submit an order for an appliance for the patient's treatment.

At block 504, the order may be submitted to the treatment system. If the treatment system is work-flow enabled (e.g., configured to use the systems and methods described herein), process 500 may continue to block 506. Otherwise, process 500 may end, which may require the doctor to manually manage remaining actions for managing the patient's treatment.

At block 506, some or all of the details regarding the order may be passed to the action management system. For example, details relating to milestones for the treatment plan may be passed.

At block 507, the details received at 506 may be applied to execute automation and/or rules. As described herein, such automation and rules may configure the various systems described herein in order to automate actions or other tasks relating to the treatment plan. Automation and rules may include updating data, propagating the updated data to relevant applications and systems, establishing and propagating trigger events, etc. An end user or a system administrator may establish rules. In some examples, the rules may be “if/then” based. For example, a rule may state that if a predetermined amount of time has elapsed, an email may be sent to the patient. Another rule may state that if a specified event occurs, a message may be sent to the doctor. Automations may similarly apply trigger conditions and responses to various connected applications and/or systems.

Returning to block 507, the automation and rules may update the patient's data, such as including order details and/or other treatment plan details. The updates may be executed at block 508. At block 508, the practice may be notified that the new order was submitted. The notification may include practice-specific data, such as data updating the patient's status, data relating to the treatment plan, etc. The updates may further trigger UI events (see, e.g., FIG. 7) and other related automations.

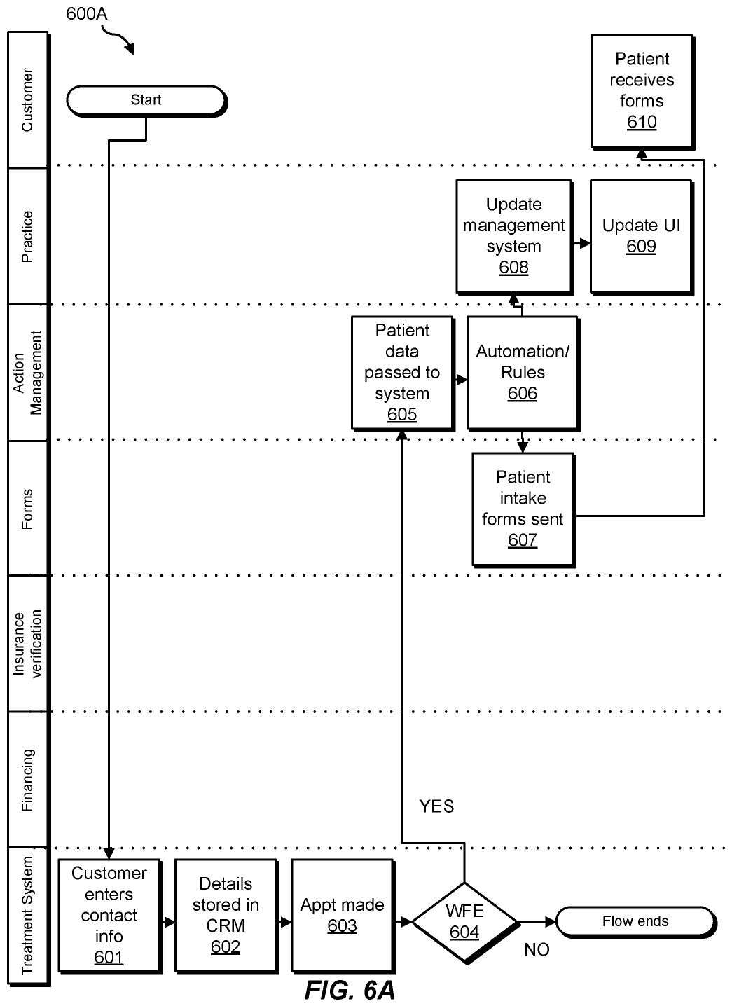

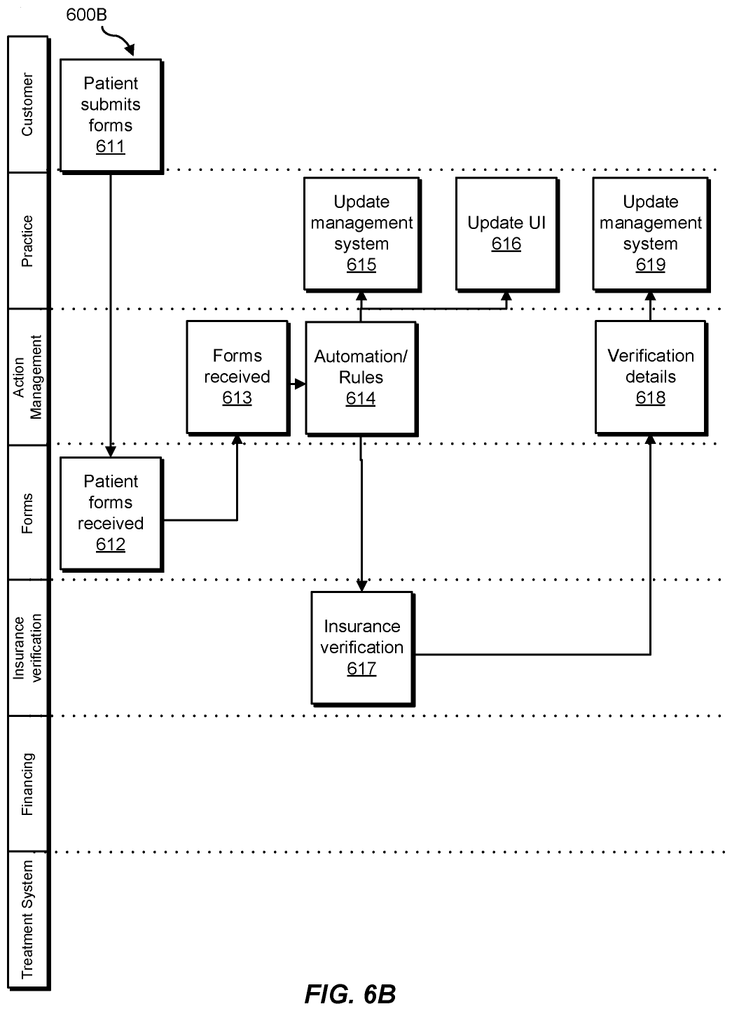

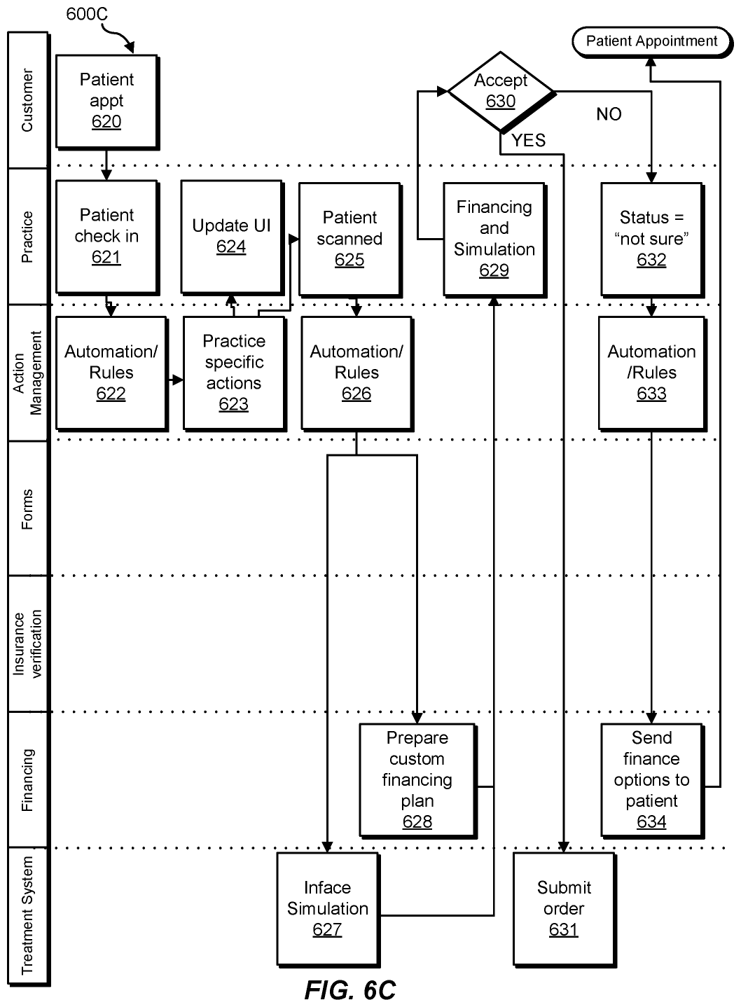

FIG. 6A shows a process 600A for initiating dental treatment of a patient. FIGS. 6A, 6B, and 6C include four rows, a customer row, a practice row, an action management row, a forms row, and insurance verification row, a financing row, and a treatment system row. The location of a block in a particular row the case the systems that may carry out the actions of that block. For example, in the customer row the customer may use a patient system to interface with the other systems in order to carry out an action. In the practice row, the dental professional system may carry out the actions of the blocks. In the action management row, an action management system or integration aggregator system there carry out the actions of the blocks. In the forms row, a SaaS system for generating and receiving information related to forms carry out the actions of the blocks. In the insurance verification row, and insurance system which may be part of the backend system may carry out the actions of the blocks. In the treatment system row, the backend system may carry out the actions of the blocks.

At block 601, the patient or consumer enters their details into a customer and patient data system. The patient may use a form such as any form provided by a SaaS system to provide their information. At block 602, the SaaS system may provide the information to the customer patient data system. In some embodiments, an action management system they retrieve order received the patient information from the SaaS system and store that information in a customer relation management system, such as the customer patient data system. At block 603, in some embodiments, the backend system may schedule an appointment for the consumer, as discussed herein. In some embodiments upon schedule an appointment for the consumer a workflow enabled check may occur at block 604. The workflow enabled check may review the data in the customer and patient data system, the appointment information, and other information related to the patient and determine whether the information is appropriate involved. If so, the process continues otherwise the process ends.

At block 605, the customer data and the appointment information are sent to the action management system. The block 605, the action management system receives the data and then at block 606 automated tasks are carried out based on rules and instructions executed by the action management system. As described herein, such automation and rules may configure the various systems described herein in order to automate actions or other tasks relating to the treatment plan. Automation and rules may include updating data, propagating the updated data to relevant applications and systems, establishing and propagating trigger events, etc. An end user or a system administrator may establish rules. In some examples, the rules may be “if/then” based. For example, a rule may state that if a predetermined amount of time has elapsed, an email may be sent to the patient. Another rule may state that if a specified event occurs, a message may be sent to the doctor. Automations may similarly apply trigger conditions and responses to various connected applications and/or systems.

For example, at block 606 the customer data in the appointment information is received as input for carrying out the rules and automation and the output of the rules and automation may be updates to the dental professional system including the generation of actions to be completed by the dental professional, and updates to data stored within the dental professional system such as treatment data patient data and other information. In some embodiments, the updates to the dental professional system may occur at block 608. In some embodiments, the rules an automation may cause the generation and send of patient intake forms at block 607. At block 608, the patient may receive the forms and begins filing them out. The intake forms may request information related to the patient's health and dental treatment. At block 610 the patient receives the forms for example on their computing device, such as a personal computer, or mobile device, such as a smartphone.

At block 609, the practice management system displays the patient details provided by the consumer at block 601 and stored in the CRM at block 602. In some embodiments, the details may include the appointment information along with the patient information.

FIG. 6B illustrates a flow diagram of a process 600B, which may continue from process 600A in FIG. 6A. At block 611, the patient may complete and submit the forms received at block 610. The forms management system may receive the patient's forms at block 612. At block 613, the action management system may be notified that the forms were received. The action management system may update the patient object to link to the patient's forms in the form management system.

At block 614, the forms received may be used to execute automation and/or rules. As described herein, such automation and rules may configure the various systems described herein in order to automate actions or other tasks relating to the treatment plan. For example, the action management system may update the management system at block 615, for instance by uploading the patient's forms to the management system, and update the UI at block 616 (see, e.g., FIG. 7), for instance by updating the patient's status to indicate the forms have been received.

Returning to block 614, another rule may trigger an automation to verify the patient's insurance, at block 617, based on the patient's details in the patient's forms. After the insurance verification system verifies the insurance details, the insurance verification system may notify the action management system with the insurance verification details at block 618. After receiving the verification details, the action management system may provide updates for the practice at block 619, for example to indicate the patient's insurance has been verified. The process 600B may then proceed to the process depicted in FIG. 6C.

FIG. 6C illustrates a flow diagram of a process 600C, which may continue from process 600B in FIG. 6B. At block 620, the patient may have an appointment scheduled, as further described herein. The patient may check in at the practice at block 621. The patient's check in may trigger automation and rules for the action management system at block 622. For example, the action management system may be updated to indicate the patient's check in and may further trigger practice specific actions at block 623.

The practice specific actions may include for example, notifying the appropriate staff member responsible for the patient intake process. Once the patient intake process is complete and updated in the action management system, the action management system may trigger a state change and alert the next staff member of the next required action. Such actions may include, for example, delivering the patient to an operatory, setting up the operatory, indicating that a scanner (e.g., dental scanning system 220) is ready, and indicating that the doctor is ready.

Once the action management system indicates the preparatory steps are ready (e.g., from block 623), at block 624 the UI may be updated to reflect the ready status. The doctor may be notified by the updated UI and may proceed to scan the patient at block 625, as described further herein. Upon completion of the patient scan, at block 626 automation and rules for the action management system may be triggered again. For example, the patient's scan data may be uploaded or otherwise linked to the appropriate systems, and the patient's status may be updated to indicate that the scan data is available.

The automation and rules may further trigger the next steps. At block 627, the treatment system may perform an inface simulation to preview the treatment. In addition, at block 628, the financing system may, using details provided by or otherwise made available through the action management system, prepare a custom financing plan for the patient to finance the treatment. At block 629, the inface simulation and the financing plan may be presented to the patient.

The patient may review the inface simulation and the financing plan at block 630. If the patient accepts, then at block 631 the order (based on the proposed treatment plan) may be submitted to the treatment system for fabrication. In some examples, the practice may receive an alert of a new bill from the treatment system regarding the submitted order. The bill may trigger other automations with other applications and/or systems, such as payment systems, etc.

However, if at block 630 the patient does not accept, then at block 632, the patient's status may be updated to “not sure” or other similar status indicating that the patient has not accepted the treatment plan. This status update may further trigger automation and rules at block 633. For example, if the patient rejects or otherwise does not accept the financing plan prepared at block 628, then at block 634 the financing system may send additional finance options to the patient. The patient may continue by scheduling a follow up appointment.

FIG. 7 depicts a user interface (“UI”) 700 that may be displayed at a doctor or staff system of a dental professional system. The interface 700 includes a plurality of columns 710 which may be referred to as swim lanes. Each of the columns 710 may include one or more cards 720. The columns 710 represent a staging the patient's treatment cycle. For example, includes cards 724 people that all dental treatment prospects that have not confirmed an appointment. Upon confirming an appointment, the person becomes a patient and their card 720 moves from the prospect columns 710 to the patient column. After the patient begins treatment, for example, by scanning their teeth, the dental professional submitting an order for a treatment plan, and receiving the treatment plan for review, the patient's card 720 may move to the right column. The right column may indicate that the patient's treatment plan is ready for review by the dental professional. Similar columns may exist for patients whose treatment plans are approved, dental appliances are sent for manufacturing, patients who are wearing aligners, patients who need follow-up appointment, and for other tasks. Although the user interface 700 shown above include swim lanes, other user interface and modules may be used to display a patient's treatment stage. For example, calendaring software may be used, task and process management software may be used for other modules and software may be used.

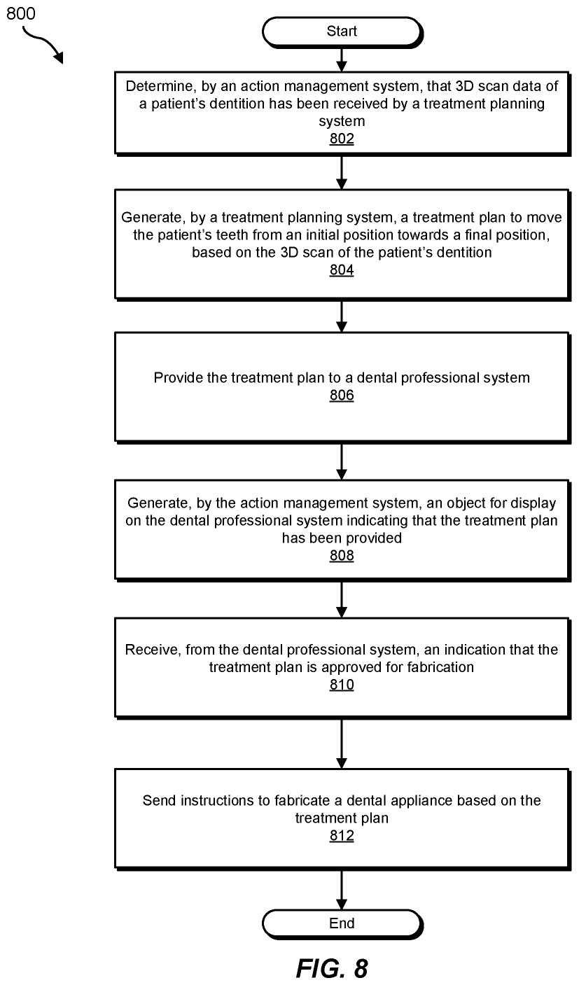

FIG. 8 depicts a process 800 for treating a patient in accordance with embodiments herein. At block 802, the process may determine, by an action management system, that 3D scan data of a patient's dentition has been received by a treatment planning system. In some embodiments, a dental professional, a patient, or other person may carry out one or more of the actions or tasks generated or provided in method 300 or any of the other methods described herein.

At block 804 the process may generate, by a treatment planning system, a treatment plan to move the patient's teeth from an initial position towards a final position, based on the 3D scan of the patient's dentition.

At block 806, the treatment plan may be provided to a dental professional system. In some embodiments, treatment plan may include moving the patient's teeth from an initial arrangement toward a target arrangement using a plurality of successive tooth-repositioning appliances. In some embodiments, the treatment plan includes a plurality of digital models of the patient's teeth, each of the plurality of digital models of the patient's teeth corresponding to a stage for the treatment plan for incrementally moving the patient's teeth from an initial arrangement toward a target arrangement.

At block 808 the process may generate, by the action management system, an object for display on the dental professional system indicating that the treatment plan has been provided. In some embodiments, at block 808 at least one of the plurality of digital models of the patient's teeth for a stage of the treatment plan is displayed at a dental professional system.

In some embodiments, after block 808 the process may include receiving, by the treatment planning system, a modification of the treatment plan. In some embodiments, the modification of the treatment plan may include a change in position or orientation of at least one of the patient's teeth for a stage of treatment.

In some embodiments, the process 800 may include generating, by the treatment planning system, a revised treatment plan. The revised treatment plan may be based on the received modification and may include a revising to a path of at least one tooth between the initial position and the final position.

In some embodiments, the process 800 may include providing the revised treatment plan to the dental professional system. In some embodiments, the process 800 may include generating, by the action management system, an object for display on the dental professional system indicting that the revised treatment plan has been provided.

At block 810, the process may receive, from the dental professional system, an indication that the treatment plan is approved for fabrication. The indication may include an approval of the treatment plan.

At block 812 instructions to fabricate a dental appliance based on the treatment plan may be sent to the fabrication system.

In some embodiments, the process 800 may include determining, by the action management system, that after a period of time, such as two weeks, that the dental appliance has not been fabricated. In some embodiments, the action management system, may generating an object for display on the dental professional system indicting that appliance has not been fabricated. In some embodiments, the process 800 may include receiving, a request to expedite fabrication from the dental professional system.

Although process 800 is presented as a sequence of steps, in some examples, the steps of process 800 may be repeated as needed.

In some embodiments, multiple dental professionals may participate in treating the patient. In some embodiments, the systems 200, 201 may include multiple dental professional systems that interact with each other and the rest of the systems. For example, in some embodiments an orthodontist may work with several general practitioner dentists and their patients. The general practitioner dentists may meet with the patient, scan their teeth provide them with treatment plan options and other dental care while the orthodontist reviews, revises and approves the treatment plan. In such an embodiment, the general practitioner dentists and their dental professional system carries out the actions and blocks associated with managing the patient, setting appointments, scanning the patient's teeth, presenting treatment plan options, and other patient associated tasks while the orthodontist and their dental professional system carries out the treatment plan review and approval for manufacturing tasks.

In some embodiments, the order for dental appliances may be placed by the general practitioner and the general practitioner pays the orthodontist for layer services. In some embodiments, the order for dental appliances may be placed by the orthodontist and the orthodontist pays the general practitioner for the referral.

### Computing System

FIG. 9 is a block diagram of an example computing system 910 capable of implementing one or more of the embodiments described and/or illustrated herein. For example, all or a portion of computing system 910 may perform and/or be a means for performing, either alone or in combination with other elements, one or more of the steps described herein (such as one or more of the steps illustrated in FIGS. 3, 4, 5, 6, and 8). All or a portion of computing system 910 may also perform and/or be a means for performing any other steps, methods, or processes described and/or illustrated herein.

Computing system 910 broadly represents any single or multi-processor computing device or system capable of executing computer-readable instructions. Examples of computing system 910 include, without limitation, workstations, laptops, client-side terminals, servers, distributed computing systems, handheld devices, or any other computing system or device. In its most basic configuration, computing system 910 may include at least one processor 914 and a system memory 916.

Processor 914 generally represents any type or form of physical processing unit (e.g., a hardware-implemented central processing unit) capable of processing data or interpreting and executing instructions. In certain embodiments, processor 914 may receive instructions from a software application or module. These instructions may cause processor 914 to perform the functions of one or more of the example embodiments described and/or illustrated herein.

System memory 916 generally represents any type or form of volatile or non-volatile storage device or medium capable of storing data and/or other computer-readable instructions. Examples of system memory 916 include, without limitation, Random Access Memory (RAM), Read Only Memory (ROM), flash memory, or any other suitable memory device. Although not required, in certain embodiments computing system 910 may include both a volatile memory unit (such as, for example, system memory 916) and a non-volatile storage device (such as, for example, primary storage device 932, as described in detail below). In one example, one or more the systems described herein may be loaded into system memory 916.

In some examples, system memory 916 may store and/or load an operating system 940 for execution by processor 914. In one example, operating system 940 may include and/or represent software that manages computer hardware and software resources and/or provides common services to computer programs and/or applications on computing system 910. Examples of operating system 940 include, without limitation, LINUX, JUNOS, MICROSOFT WINDOWS, WINDOWS MOBILE, MAC OS, APPLE'S IOS, UNIX, GOOGLE CHROME OS, GOOGLE'S ANDROID, SOLARIS, variations of one or more of the same, and/or any other suitable operating system.

In certain embodiments, example computing system 910 may also include one or more components or elements in addition to processor 914 and system memory 916. For example, as illustrated in FIG. 9, computing system 910 may include a memory controller 918, an Input/Output (I/O) controller 920, and a communication interface 922, each of which may be interconnected via a communication infrastructure 912. Communication infrastructure 912 generally represents any type or form of infrastructure capable of facilitating communication between one or more components of a computing device. Examples of communication infrastructure 912 include, without limitation, a communication bus (such as an Industry Standard Architecture (ISA), Peripheral Component Interconnect (PCI), PCI Express (PCIe), or similar bus) and a network.

Memory controller 918 generally represents any type or form of device capable of handling memory or data or controlling communication between one or more components of computing system 910. For example, in certain embodiments memory controller 918 may control communication between processor 914, system memory 916, and I/O controller 920 via communication infrastructure 912.

I/O controller 920 generally represents any type or form of module capable of coordinating and/or controlling the input and output functions of a computing device. For example, in certain embodiments I/O controller 920 may control or facilitate transfer of data between one or more elements of computing system 910, such as processor 914, system memory 916, communication interface 922, display adapter 926, input interface 930, and storage interface 934.

As illustrated in FIG. 9, computing system 910 may also include at least one display device 924 coupled to I/O controller 920 via a display adapter 926. Display device 924 generally represents any type or form of device capable of visually displaying information forwarded by display adapter 926. Similarly, display adapter 926 generally represents any type or form of device configured to forward graphics, text, and other data from communication infrastructure 912 (or from a frame buffer, as known in the art) for display on display device 924.

As illustrated in FIG. 9, example computing system 910 may also include at least one input device 928 coupled to I/O controller 920 via an input interface 930. Input device 928 generally represents any type or form of input device capable of providing input, either computer or human generated, to example computing system 910. Examples of input device 928 include, without limitation, a keyboard, a pointing device, a speech recognition device, variations or combinations of one or more of the same, and/or any other input device.

Additionally or alternatively, example computing system 910 may include additional I/O devices. For example, example computing system 910 may include I/O device 936. In this example, I/O device 936 may include and/or represent a user interface that facilitates human interaction with computing system 910. Examples of I/O device 936 include, without limitation, a computer mouse, a keyboard, a monitor, a printer, a modem, a camera, a scanner, a microphone, a touchscreen device, variations or combinations of one or more of the same, and/or any other I/O device.

Communication interface 922 broadly represents any type or form of communication device or adapter capable of facilitating communication between example computing system 910 and one or more additional devices. For example, in certain embodiments communication interface 922 may facilitate communication between computing system 910 and a private or public network including additional computing systems. Examples of communication interface 922 include, without limitation, a wired network interface (such as a network interface card), a wireless network interface (such as a wireless network interface card), a modem, and any other suitable interface. In at least one embodiment, communication interface 922 may provide a direct connection to a remote server via a direct link to a network, such as the Internet. Communication interface 922 may also indirectly provide such a connection through, for example, a local area network (such as an Ethernet network), a personal area network, a telephone or cable network, a cellular telephone connection, a satellite data connection, or any other suitable connection.

In certain embodiments, communication interface 922 may also represent a host adapter configured to facilitate communication between computing system 910 and one or more additional network or storage devices via an external bus or communications channel. Examples of host adapters include, without limitation, Small Computer System Interface (SCSI) host adapters, Universal Serial Bus (USB) host adapters, Institute of Electrical and Electronics Engineers (IEEE) 1394 host adapters, Advanced Technology Attachment (ATA), Parallel ATA (PATA), Serial ATA (SATA), and External SATA (eSATA) host adapters, Fibre Channel interface adapters, Ethernet adapters, or the like. Communication interface 922 may also allow computing system 910 to engage in distributed or remote computing. For example, communication interface 922 may receive instructions from a remote device or send instructions to a remote device for execution.

In some examples, system memory 916 may store and/or load a network communication program 938 for execution by processor 914. In one example, network communication program 938 may include and/or represent software that enables computing system 910 to establish a network connection 942 with another computing system (not illustrated in FIG. 9) and/or communicate with the other computing system by way of communication interface 922. In this example, network communication program 938 may direct the flow of outgoing traffic that is sent to the other computing system via network connection 942. Additionally or alternatively, network communication program 938 may direct the processing of incoming traffic that is received from the other computing system via network connection 942 in connection with processor 914.

Although not illustrated in this way in FIG. 9, network communication program 938 may alternatively be stored and/or loaded in communication interface 922. For example, network communication program 938 may include and/or represent at least a portion of software and/or firmware that is executed by a processor and/or Application Specific Integrated Circuit (ASIC) incorporated in communication interface 922.

As illustrated in FIG. 9, example computing system 910 may also include a primary storage device 932 and a backup storage device 933 coupled to communication infrastructure 912 via a storage interface 934. Storage devices 932 and 933 generally represent any type or form of storage device or medium capable of storing data and/or other computer-readable instructions. For example, storage devices 932 and 933 may be a magnetic disk drive (e.g., a so-called hard drive), a solid state drive, a floppy disk drive, a magnetic tape drive, an optical disk drive, a flash drive, or the like. Storage interface 934 generally represents any type or form of interface or device for transferring data between storage devices 932 and 933 and other components of computing system 910. In one example, patient data may be stored and/or loaded in primary storage device 932.

In certain embodiments, storage devices 932 and 933 may be configured to read from and/or write to a removable storage unit configured to store computer software, data, or other computer-readable information. Examples of suitable removable storage units include, without limitation, a floppy disk, a magnetic tape, an optical disk, a flash memory device, or the like. Storage devices 932 and 933 may also include other similar structures or devices for allowing computer software, data, or other computer-readable instructions to be loaded into computing system 910. For example, storage devices 932 and 933 may be configured to read and write software, data, or other computer-readable information. Storage devices 932 and 933 may also be a part of computing system 910 or may be a separate device accessed through other interface systems.

Many other devices or subsystems may be connected to computing system 910. Conversely, all of the components and devices illustrated in FIG. 9 need not be present to practice the embodiments described and/or illustrated herein. The devices and subsystems referenced above may also be interconnected in different ways from that shown in FIG. 9. Computing system 910 may also employ any number of software, firmware, and/or hardware configurations. For example, one or more of the example embodiments disclosed herein may be encoded as a computer program (also referred to as computer software, software applications, computer-readable instructions, or computer control logic) on a computer-readable medium. The term “computer-readable medium,” as used herein, generally refers to any form of device, carrier, or medium capable of storing or carrying computer-readable instructions. Examples of computer-readable media include, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

The computer-readable medium containing the computer program may be loaded into computing system 910. All or a portion of the computer program stored on the computer-readable medium may then be stored in system memory 916 and/or various portions of storage devices 932 and 933. When executed by processor 914, a computer program loaded into computing system 910 may cause processor 914 to perform and/or be a means for performing the functions of one or more of the example embodiments described and/or illustrated herein. Additionally or alternatively, one or more of the example embodiments described and/or illustrated herein may be implemented in firmware and/or hardware. For example, computing system 910 may be configured as an Application Specific Integrated Circuit (ASIC) adapted to implement one or more of the example embodiments disclosed herein.

FIG. 10 is a block diagram of an example network architecture 1000 in which client systems 1010, 1020, and 1030 and servers 1040 and 1045 may be coupled to a network 1050. As detailed above, all or a portion of network architecture 1000 may perform and/or be a means for performing, either alone or in combination with other elements, one or more of the steps disclosed herein (such as one or more of the steps illustrated in FIGS. 3, 4, 5, 6, and 8). All or a portion of network architecture 1000 may also be used to perform and/or be a means for performing other steps and features set forth in the instant disclosure.

Client systems 1010, 1020, and 1030 generally represent any type or form of computing device or system, such as example computing system 910 in FIG. 9. Similarly, servers 1040 and 1045 generally represent computing devices or systems, such as application servers or database servers, configured to provide various database services and/or run certain software applications. Network 1050 generally represents any telecommunication or computer network including, for example, an intranet, a WAN, a LAN, a PAN, or the Internet. In one example, client systems 1010, 1020, and/or 1030 and/or servers 1040 and/or 1045 may include all or a portion of system 200 from FIG. 2A.

As illustrated in FIG. 10, one or more storage devices 1060(1)-(N) may be directly attached to server 1040. Similarly, one or more storage devices 1070(1)-(N) may be directly attached to server 1045. Storage devices 1060(1)-(N) and storage devices 1070(1)-(N) generally represent any type or form of storage device or medium capable of storing data and/or other computer-readable instructions. In certain embodiments, storage devices 1060(1)-(N) and storage devices 1070(1)-(N) may represent Network-Attached Storage (NAS) devices configured to communicate with servers 1040 and 1045 using various protocols, such as Network File System (NFS), Server Message Block (SMB), or Common Internet File System (CIFS).

Servers 1040 and 1045 may also be connected to a Storage Area Network (SAN) fabric 1080. SAN fabric 1080 generally represents any type or form of computer network or architecture capable of facilitating communication between a plurality of storage devices. SAN fabric 1080 may facilitate communication between servers 1040 and 1045 and a plurality of storage devices 1090(1)-(N) and/or an intelligent storage array 1095. SAN fabric 1080 may also facilitate, via network 1050 and servers 1040 and 1045, communication between client systems 1010, 1020, and 1030 and storage devices 1090(1)-(N) and/or intelligent storage array 1095 in such a manner that devices 1090(1)-(N) and array 1095 appear as locally attached devices to client systems 1010, 1020, and 1030. As with storage devices 1060(1)-(N) and storage devices 1070(1)-(N), storage devices 1090(1)-(N) and intelligent storage array 1095 generally represent any type or form of storage device or medium capable of storing data and/or other computer-readable instructions.

In certain embodiments, and with reference to example computing system 910 of FIG. 9, a communication interface, such as communication interface 922 in FIG. 9, may be used to provide connectivity between each client system 1010, 1020, and 1030 and network 1050. Client systems 1010, 1020, and 1030 may be able to access information on server 1040 or 1045 using, for example, a web browser or other client software. Such software may allow client systems 1010, 1020, and 1030 to access data hosted by server 1040, server 1045, storage devices 1060(1)-(N), storage devices 1070(1)-(N), storage devices 1090(1)-(N), or intelligent storage array 1095. Although FIG. 10 depicts the use of a network (such as the Internet) for exchanging data, the embodiments described and/or illustrated herein are not limited to the Internet or any particular network-based environment.

In at least one embodiment, all or a portion of one or more of the example embodiments disclosed herein may be encoded as a computer program and loaded onto and executed by server 1040, server 1045, storage devices 1060(1)-(N), storage devices 1070(1)-(N), storage devices 1090(1)-(N), intelligent storage array 1095, or any combination thereof. All or a portion of one or more of the example embodiments disclosed herein may also be encoded as a computer program, stored in server 1040, run by server 1045, and distributed to client systems 1010, 1020, and 1030 over network 1050.

As detailed above, computing system 910 and/or one or more components of network architecture 1000 may perform and/or be a means for performing, either alone or in combination with other elements, one or more steps of an example method for virtual care.

While the foregoing disclosure sets forth various embodiments using specific block diagrams, flowcharts, and examples, each block diagram component, flowchart step, operation, and/or component described and/or illustrated herein may be implemented, individually and/or collectively, using a wide range of hardware, software, or firmware (or any combination thereof) configurations. In addition, any disclosure of components contained within other components should be considered example in nature since many other architectures can be implemented to achieve the same functionality.

In some examples, all or a portion of example system 200 or system 201 in FIGS. 2A and 2B may represent portions of a cloud-computing or network-based environment. Cloud-computing environments may provide various services and applications via the Internet. These cloud-based services (e.g., software as a service, platform as a service, infrastructure as a service, etc.) may be accessible through a web browser or other remote interface. Various functions described herein may be provided through a remote desktop environment or any other cloud-based computing environment.

In various embodiments, all or a portion of example system 200 or system 201 in FIG. 2A or 2B may facilitate multi-tenancy within a cloud-based computing environment. In other words, the software modules described herein may configure a computing system (e.g., a server) to facilitate multi-tenancy for one or more of the functions described herein. For example, one or more of the software modules described herein may program a server to enable two or more clients (e.g., customers) to share an application that is running on the server. A server programmed in this manner may share an application, operating system, processing system, and/or storage system among multiple customers (i.e., tenants). One or more of the modules described herein may also partition data and/or configuration information of a multi-tenant application for each customer such that one customer cannot access data and/or configuration information of another customer.

According to various embodiments, all or a portion of example system 200 in FIG. 2A may be implemented within a virtual environment. For example, the modules and/or data described herein may reside and/or execute within a virtual machine. As used herein, the term “virtual machine” generally refers to any operating system environment that is abstracted from computing hardware by a virtual machine manager (e.g., a hypervisor). Additionally or alternatively, the modules and/or data described herein may reside and/or execute within a virtualization layer. As used herein, the term “virtualization layer” generally refers to any data layer and/or application layer that overlays and/or is abstracted from an operating system environment. A virtualization layer may be managed by a software virtualization solution (e.g., a file system filter) that presents the virtualization layer as though it were part of an underlying base operating system. For example, a software virtualization solution may redirect calls that are initially directed to locations within a base file system and/or registry to locations within a virtualization layer.

In some examples, all or a portion of example system 200 in FIG. 2A may represent portions of a mobile computing environment. Mobile computing environments may be implemented by a wide range of mobile computing devices, including mobile phones, tablet computers, e-book readers, personal digital assistants, wearable computing devices (e.g., computing devices with a head-mounted display, smartwatches, etc.), and the like. In some examples, mobile computing environments may have one or more distinct features, including, for example, reliance on battery power, presenting only one foreground application at any given time, remote management features, touchscreen features, location and movement data (e.g., provided by Global Positioning Systems, gyroscopes, accelerometers, etc.), restricted platforms that restrict modifications to system-level configurations and/or that limit the ability of third-party software to inspect the behavior of other applications, controls to restrict the installation of applications (e.g., to only originate from approved application stores), etc. Various functions described herein may be provided for a mobile computing environment and/or may interact with a mobile computing environment.

In addition, all or a portion of example system 200 in FIG. 2A may represent portions of, interact with, consume data produced by, and/or produce data consumed by one or more systems for information management. As used herein, the term “information management” may refer to the protection, organization, and/or storage of data. Examples of systems for information management may include, without limitation, storage systems, backup systems, archival systems, replication systems, high availability systems, data search systems, virtualization systems, and the like.

In some embodiments, all or a portion of example system 200 in FIG. 2A may represent portions of, produce data protected by, and/or communicate with one or more systems for information security. As used herein, the term “information security” may refer to the control of access to protected data. Examples of systems for information security may include, without limitation, systems providing managed security services, data loss prevention systems, identity authentication systems, access control systems, encryption systems, policy compliance systems, intrusion detection and prevention systems, electronic discovery systems, and the like.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

Unless otherwise noted, the terms “connected to” and “coupled to” (and their derivatives), as used in the specification and claims, are to be construed as permitting both direct and indirect (i.e., via other elements or components) connection. In addition, the terms “a” or “an,” as used in the specification and claims, are to be construed as meaning “at least one of” Finally, for ease of use, the terms “including” and “having” (and their derivatives), as used in the specification and claims, are interchangeable with and shall have the same meaning as the word “comprising.

The processor as disclosed herein can be configured with instructions to perform any one or more steps of any method as disclosed herein.

It will be understood that although the terms “first,” “second,” “third”, etc. may be used herein to describe various layers, elements, components, regions or sections without referring to any particular order or sequence of events. These terms are merely used to distinguish one layer, element, component, region or section from another layer, element, component, region or section. A first layer, element, component, region or section as described herein could be referred to as a second layer, element, component, region or section without departing from the teachings of the present disclosure.

As used herein, the term “or” is used inclusively to refer items in the alternative and in combination.

As used herein, characters such as numerals refer to like elements.

The present disclosure includes the following numbered clauses.

***

Clause 1. A method for preparing for and providing dental treatment, the method comprising: determining, by an action management system, that 3D scan data of a patient's dentition has been received by a treatment planning system; generating, by a treatment planning system, a treatment plan to move the patient's teeth from an initial position towards a final position, based on the 3D scan of the patient's dentition; providing the treatment plan to a dental professional system; generating, by the action management system, an object for display on the dental professional system indicating that the treatment plan has been provided; receiving, from the dental professional system, an indication that the treatment plan is approved for fabrication; sending instructions to fabricate a dental appliance based on the treatment plan.

Clause 2. The method of clause 1, wherein the treatment plan includes moving the patient's teeth from an initial arrangement toward a target arrangement using a plurality of successive tooth-repositioning appliances.

Clause 3. The method of clause 1, wherein the treatment plan includes a plurality of digital models of the patient's teeth, each of the plurality of digital models of the patient's teeth corresponding to a stage for the treatment plan for incrementally moving the patient's teeth from an initial arrangement toward a target arrangement.

Clause 4. The method of clause 3, further comprising: displaying at least one of the plurality of digital models of the patient's teeth for a stage of the treatment plan.

Clause 5. The method of clause 4, further comprising: receiving, by the treatment planning system, a modification of the treatment plan.

Clause 6. The method of clause 5, wherein: the modification of the treatment plan includes a change in position or orientation of at least one of the patient's teeth for a stage of treatment.

Clause 7. The method of clause 4, further comprising: generating, by the treatment planning system, a revised treatment plan; providing the revised treatment plan to the dental professional system; and generating, by the action management system, an object for display on the dental professional system indicting that the revised treatment plan has been provided.

Clause 8. The method of clause 1, further comprising: displaying the object for display on the dental professional system.

Clause 9. The method of clause 1, further comprising: determining, by the action management system, that after a first time period, the dental appliance has not been fabricated; and generating, by the action management system, an object for display on the dental professional system indicting that appliance has not been fabricated.

Clause 10. The method of clause 9, further comprising: receiving, in response to the generating by the action management system, the object for display on the dental professional system indicting that appliance has not been fabricated, a request to expedite fabrication.

Clause 11. The method of clause 1, further comprising: receiving patient data into a patient and consumer data system.

Clause 12. The method of clause 11, further comprising: providing the patient data to a dental professional system.

Clause 13. The method of clause 12, wherein patient data includes patient one or more of name, address, or medical history.

Clause 14. The method of clause 12, further comprising: determining, buy the action management system, the dental professional to treat the patient.

Clause 15. The method of clause 14, further comprising: displaying, on the dental professional system, the object an object appears including the patient data.

Clause 16. The method of clause 15, further comprising: generating automatically, at the dental professional system, an electronic patient file based on the received patient data.

Clause 17. The method of clause 16, further comprising: automatically initiating a sending, by the action management system, a communication to the patient regarding their treatment.

Clause 18. The method of clause 16, further comprising: automatically initiating a sending, by the action management system, of a request for additional information from the patient.

Clause 19. The method of clause 18, further comprising: receiving the additional information from the patient; adding the additional information to the patient data; and adding the additional information to the electronic patient file at the dental professional system.

Clause 20. The method of clause 19, further comprising: updating a state of the patient file from an intake state to treatment state after adding the additional information to the electronic patient file at the dental professional system.

Clause 21. The method of clause 20, further comprising: generating, by the action management system, treatment actions.

Clause 22. The method of clause 21, wherein the treatment actions include one or more of setting up the operatory, generating a scan of the patient's dentition, and sending the scan to a treatment planning system.

Clause 23. The method of clause 22, wherein a first device of the dental professional system facilitates the adding the additional information to the electronic patient file and a second device of the dental professional system facilitates generating the treatment actions.

Clause 24. The method of clause 23, wherein determining, by an action management system, that 3D scan data of a patient's dentition has been received by a treatment planning system including determining that an order for dental treatment has been generated.

Clause 25. The method of clause 24, further comprising: sending a confirmation of the order to dental professional system; and generating, by the action management system, an action for a third device of the dental professional system based on the sending of the confirmation.

Clause 26. The method of clause 17, further comprising: scheduling, at the practice management system, an appointment for treatment; and generating, in response to the scheduling, at the action management system, treatment actions for the dental professional.

Clause 27. The method of clause 26, wherein the actions include one or more of generating 2D images, x-rays, three-dimensional scans of the patient's dentition, simulation of dental treatment, or other dental treatment actions.

Clause 28. The method of clause 27, further comprising, displaying the actions at the dental professional system.

Clause 29. The method of clause 27, further comprising determining that an action is complete; and removing the action from the dental professional system.

Clause 30. The method of clause 29, wherein determining that an corresponding action is complete occurs in response to generation of the 2D images, the x-rays, the three-dimensional scans of the patient's dentition, or the simulation of dental treatment.

Clause 31. The method of clause 29, wherein determining that an corresponding action is complete occurs in response to receiving the 2D images, the x-rays, the three-dimensional scans of the patient's dentition, or the simulation of dental treatment.

Clause 32. The method of clause 1, further comprising: fabricating the dental appliance.

Clause 33. The method of clause 32, wherein the dental appliance is an aligner, crown, or bridge.

Clause 34. The method of clause 32, wherein the dental appliance is directly fabricated.

Clause 35. The method of clause 32, wherein the dental appliance is indirectly fabricated.

Clause 36. The method of clause 35, further comprising directly fabricating a mold for the dental appliance.

Clause 37. The method of clause 36, further comprising, forming the dental appliance over or in the mold.

Clause 38. The method of clause 37, further comprising, determining that the dental appliance has been provided to the patient.

Clause 39. The method of clause 38, further comprising: generating, by the action management system, treatment actions for the treatment of the patient.

Clause 40. The method of clause 39, wherein the treatment actions include instructions for dates on which the patient is progress to the next stage of treatment.

Clause 41. The method of clause 40, wherein progressing to the next stage of treatment includes wearing a new aligner.

Clause 42. A system for preparing for and providing dental treatment, the system comprising: a processor; and memory comprising instructions that when executed by the processor cause the system to carry out a method comprising: determining, by an action management system, that 3D scan data of a patient's dentition has been received by a treatment planning system; generating, by a treatment planning system, a treatment plan to move the patient's teeth from an initial position towards a final position, based on the 3D scan of the patient's dentition; providing the treatment plan to a dental professional system; generating, by the action management system, an object for display on the dental professional system indicating that the treatment plan has been provided; receiving, from the dental professional system, an indication that the treatment plan is approved for fabrication; sending instructions to fabricate a dental appliance based on the treatment plan.

Clause 43. The system of clause 42, wherein the treatment plan includes moving the patient's teeth from an initial arrangement toward a target arrangement using a plurality of successive tooth-repositioning appliances.

Clause 44. The system of clause 42, wherein the treatment plan includes a plurality of digital models of the patient's teeth, each of the plurality of digital models of the patient's teeth corresponding to a stage for the treatment plan for incrementally moving the patient's teeth from an initial arrangement toward a target arrangement.

Clause 45. The system of clause 44, wherein the memory comprising instructions that when executed by the processor cause the system to carry out the method, the method further comprising: displaying at least one of the plurality of digital models of the patient's teeth for a stage of the treatment plan.

Clause 46. The system of clause 44, wherein the memory comprising instructions that when executed by the processor cause the system to carry out the method, the method further comprising: receiving, by the treatment planning system, a modification of the treatment plan.

Clause 47. The system of clause 44, wherein: the modification of the treatment plan includes a change in position or orientation of at least one of the patient's teeth for a stage of treatment.

Clause 48. The system of clause 44, wherein the memory comprising instructions that when executed by the processor cause the system to carry out the method, the method further comprising: generating, by the treatment planning system, a revised treatment plan; providing the revised treatment plan to the dental professional system; and generating, by the action management system, an object for display on the dental professional system indicting that the revised treatment plan has been provided.

Clause 49. The system of clause 42, wherein the memory comprising instructions that when executed by the processor cause the system to carry out the method, the method further comprising: displaying the object for display on the dental professional system.

Clause 50. The system of clause 42, wherein the memory comprising instructions that when executed by the processor cause the system to carry out the method, the method further comprising: determining, by the action management system, that after a first time period, the dental appliance has not been fabricated; and generating, by the action management system, an object for display on the dental professional system indicting that appliance has not been fabricated.

Clause 51. The system of clause 50, wherein the memory comprising instructions that when executed by the processor cause the system to carry out the method, the method further comprising: receiving, in response to the generating by the action management system, the object for display on the dental professional system indicting that appliance has not been fabricated, a request to expedite fabrication.

Clause 52. The system of clause 42, wherein the memory comprising instructions that when executed by the processor cause the system to carry out the method, the method further comprising: receiving patient data into a patient and consumer data system.

Clause 53. The system of clause 52, wherein the memory comprising instructions that when executed by the processor cause the system to carry out the method, the method further comprising: providing the patient data to a dental professional system.

Clause 54. The system of clause 53, wherein patient data includes patient one or more of name, address, or medical history.

Clause 55. The system of clause 53, wherein the memory comprising instructions that when executed by the processor cause the system to carry out the method, the method further comprising: determining, buy the action management system, the dental professional to treat the patient.

Clause 56. The system of clause 55, wherein the memory comprising instructions that when executed by the processor cause the system to carry out the method, the method further comprising: displaying, on the dental professional system, the object an object appears including the patient data.

Clause 57. The system of clause 56, wherein the memory comprising instructions that when executed by the processor cause the system to carry out the method, the method further comprising: generating automatically, at the dental professional system, an electronic patient file based on the received patient data.

Clause 58. The system of clause 57, wherein the memory comprising instructions that when executed by the processor cause the system to carry out the method, the method further comprising: automatically initiating a sending, by the action management system, a communication to the patient regarding their treatment.

Clause 59. The system of clause 57, wherein the memory comprising instructions that when executed by the processor cause the system to carry out the method, the method further comprising: automatically initiating a sending, by the action management system, of a request for additional information from the patient.

Clause 60. The system of clause 59, wherein the memory comprising instructions that when executed by the processor cause the system to carry out the method, the method further comprising: receiving the additional information from the patient; adding the additional information to the patient data; and adding the additional information to the electronic patient file at the dental professional system.

Clause 61. The system of clause 60, wherein the memory comprising instructions that when executed by the processor cause the system to carry out the method, the method further comprising: updating a state of the patient file from an intake state to treatment state after adding the additional information to the electronic patient file at the dental professional system.

Clause 62. The system of clause 61, wherein the memory comprising instructions that when executed by the processor cause the system to carry out the method, the method further comprising: generating, by the action management system, treatment actions.

Clause 63. The system of clause 62, wherein the treatment actions include one or more of setting up the operatory, generating a scan of the patient's dentition, and sending the scan to a treatment planning system.

Clause 64. The system of clause 63, wherein a first device of the dental professional system facilitates the adding the additional information to the electronic patient file and a second device of the dental professional system facilitates generating the treatment actions.

Clause 65. The system of clause 64, wherein determining, by an action management system, that 3D scan data of a patient's dentition has been received by a treatment planning system including determining that an order for dental treatment has been generated.

Clause 66. The system of clause 65, wherein the memory comprising instructions that when executed by the processor cause the system to carry out the method, the method further comprising: sending a confirmation of the order to dental professional system; and generating, by the action management system, an action for a third device of the dental professional system based on the sending of the confirmation.

Clause 67. The system of clause 58, wherein the memory comprising instructions that when executed by the processor cause the system to carry out the method, the method further comprising: scheduling, at the practice management system, an appointment for treatment; and generating, in response to the scheduling, at the action management system, treatment actions for the dental professional.

Clause 68. The system of clause 67, wherein the actions include one or more of generating 2D images, x-rays, three-dimensional scans of the patient's dentition, simulation of dental treatment, or other dental treatment actions.

Clause 69. The system of clause 68, wherein the memory comprising instructions that when executed by the processor cause the system to carry out the method, the method further comprising, displaying the actions at the dental professional system.

Clause 70. The system of clause 68, wherein the memory comprising instructions that when executed by the processor cause the system to carry out the method, the method further comprising determining that an action is complete; and removing the action from the dental professional system.

Clause 71. The system of clause 70, wherein determining that a corresponding action is complete occurs in response to generation of the 2D images, the x-rays, the three-dimensional scans of the patient's dentition, or the simulation of dental treatment.

Clause 72. The system of clause 29, wherein determining that a corresponding action is complete occurs in response to receiving the 2D images, the x-rays, the three-dimensional scans of the patient's dentition, or the simulation of dental treatment.

Clause 73. The system of clause 42, wherein the memory comprising instructions that when executed by the processor cause the system to carry out the method, the method further comprising: fabricating the dental appliance.

Clause 74. The system of clause 73, wherein the dental appliance is an aligner, crown, or bridge.

Clause 75. The system of clause 73, wherein the dental appliance is directly fabricated.

Clause 76. The system of clause 73, wherein the dental appliance is indirectly fabricated.

Clause 77. The system of clause 76, wherein the memory comprising instructions that when executed by the processor cause the system to carry out the method, the method further comprising directly fabricating a mold for the dental appliance.

Clause 78. The system of clause 77, wherein the memory comprising instructions that when executed by the processor cause the system to carry out the method, the method further comprising, forming the dental appliance over or in the mold.

Clause 79. The system of clause 78, wherein the memory comprising instructions that when executed by the processor cause the system to carry out the method, the method further comprising, determining that the dental appliance has been provided to the patient.

Clause 80. The system of clause 79, wherein the memory comprising instructions that when executed by the processor cause the system to carry out the method, the method further comprising: generating, by the action management system, treatment actions for the treatment of the patient.

Clause 81. The system of clause 80, wherein the treatment actions include instructions for dates on which the patient is progress to the next stage of treatment.

Clause 82. The system of clause 81, wherein progressing to the next stage of treatment includes wearing a new aligner.

Embodiments of the present disclosure have been shown and described as set forth herein and are provided by way of example only. One of ordinary skill in the art will recognize numerous adaptations, changes, variations and substitutions without departing from the scope of the present disclosure. Several alternatives and combinations of the embodiments disclosed herein may be utilized without departing from the scope of the present disclosure and the inventions disclosed herein. Therefore, the scope of the presently disclosed inventions shall be defined solely by the scope of the appended claims and the equivalents thereof

What is claimed is:

1. A method for preparing for and providing dental treatment, the method comprising:
determining, by an action management system, that 3D scan data of a patient's dentition has been received by a treatment planning system;
generating, by a treatment planning system, a treatment plan to move the patient's teeth from an initial position towards a final position, based on the 3D scan of the patient's dentition;
providing the treatment plan to a dental professional system;
generating, by the action management system, an object for display on the dental professional system indicating that the treatment plan has been provided;
receiving, from the dental professional system, an indication that the treatment plan is approved for fabrication;
sending instructions to fabricate a dental appliance based on the treatment plan.

2. The method of claim 1, wherein the treatment plan includes moving the patient's teeth from an initial arrangement toward a target arrangement using a plurality of successive tooth-repositioning appliances.

3. The method of claim 1, wherein the treatment plan includes a plurality of digital models of the patient's teeth, each of the plurality of digital models of the patient's teeth corresponding to a stage for the treatment plan for incrementally moving the patient's teeth from an initial arrangement toward a target arrangement.

4. The method of claim 3, further comprising:
displaying at least one of the plurality of digital models of the patient's teeth for a stage of the treatment plan.

5. The method of claim 4, further comprising:
receiving, by the treatment planning system, a modification of the treatment plan.

6. The method of claim 5, wherein:
the modification of the treatment plan includes a change in position or orientation of at least one of the patient's teeth for a stage of treatment.

7. The method of claim 4, further comprising:
generating, by the treatment planning system, a revised treatment plan;
providing the revised treatment plan to the dental professional system; and
generating, by the action management system, an object for display on the dental professional system indicting that the revised treatment plan has been provided.

8. The method of claim 1, further comprising:
displaying the object for display on the dental professional system.

9. The method of claim 1, further comprising:
determining, by the action management system, that after a first time period, the dental appliance has not been fabricated; and
generating, by the action management system, an object for display on the dental professional system indicting that appliance has not been fabricated.

10. The method of claim 9, further comprising:
receiving, in response to the generating by the action management system, the object for display on the dental professional system indicting that appliance has not been fabricated, a request to expedite fabrication.

11. The method of claim 1, further comprising:
receiving patient data into a patient and consumer data system.

12. The method of claim 11, further comprising:
providing the patient data to a dental professional system.

13. The method of claim 12, wherein patient data includes patient one or more of name, address, or medical history.

14. The method of claim 12, further comprising:
determining, buy the action management system, the dental professional to treat the patient.

15. The method of claim 14, further comprising:
displaying, on the dental professional system, the object an object appears including the patient data.

16. The method of claim 15, further comprising:
generating automatically, at the dental professional system, an electronic patient file based on the received patient data.

17. The method of claim 16, further comprising:
automatically initiating a sending, by the action management system, a communication to the patient regarding their treatment.

18. The method of claim 16, further comprising:
automatically initiating a sending, by the action management system, of a request for additional information from the patient.

19. The method of claim 18, further comprising:
receiving the additional information from the patient;
adding the additional information to the patient data; and
adding the additional information to the electronic patient file at the dental professional system.

20. The method of claim 19, further comprising:
updating a state of the patient file from an intake state to treatment state after adding the additional information to the electronic patient file at the dental professional system.
